SEQUENCE LISTING (18 sequences)

SEQ ID NO: 1 - DNA, 1599 bp, Aspergillus fumigatus
atgctggcctccaccttctcctaccgcatgtacaagaccgcgctcatcctggccgcccttctgggctctggccaggctcagcaggtcggtacttcccaggcggaagtgcatccgtccatgacctggcagagctgcacggctggcggcagctgcaccaccaacaacggcaaggtggtcatcgacgcgaactggcgttgggtgcacaaagtcggcgactacaccaactgctacaccggcaacacctgggacacgactatctgccctgacgatgcgacctgcgcatccaactgcgcccttgagggtgccaactacgaatccacctatggtgtgaccgccagcggcaattccctccgcctcaacttcgtcaccaccagccagcagaagaacattggctcgcgtctgtacatgatgaaggacgactcgacctacgagatgtttaagctgctgaaccaggagttcaccttcgatgtcgatgtctccaacctcccctgcggtctcaacggtgctctgtactttgtcgccatggacgccgacggtggcatgtccaagtacccaaccaacaaggccggtgccaagtacggtactggatactgtgactcgcagtgccctcgcgacctcaagttcatcaacggtcaggccaacgtcgaagggtggcagccctcctccaacgatgccaatgcgggtaccggcaaccacgggtcctgctgcgcggagatggatatctgggaggccaacagcatctccacggccttcaccccccatccgtgcgacacgcccggccaggtgatgtgcaccggtgatgcctgcggtggcacctacagctccgaccgctacggcggcacctgcgaccccgacggatgtgatttcaactccttccgccagggcaacaagaccttctacggccctggcatgaccgtcgacaccaagagcaagtttaccgtcgtcacccagttcatcaccgacgacggcacctccagcggcaccctcaaggagatcaagcgcttctacgtgcagaacggcaaggtgatccccaactcggagtcgacctggaccggcgtcagcggcaactccatcaccaccgagtactgcaccgcccagaagagcctgttccaggaccagaacgtcttcgaaaagcacggcggcctcgagggcatgggtgctgccctcgcccagggtatggttctcgtcatgtccctgtgggatgatcactcggccaacatgctctggctcgacagcaactacccgaccactgcctcttccaccactcccggcgtcgcccgtggtacctgcgacatctcctccggcgtccctgcggatgtcgaggcgaaccaccccgacgcctacgtcgtctactccaacatcaaggtcggccccatcggctcgaccttcaacagcggtggctcgaaccccggtggcggaaccaccacgacaactaccacccagcctactaccaccacgaccacggctggaaaccctggcggcaccggagtcgcacagcactatggccagtgtggtggaatcggatggaccggacccacaacctgtgccagcccttatacctgccagaagctgaatgattattactctcagtgcctgtag

SEQ ID NO: 2 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 3 - DNA, 1713 bp, Aspergillus fumigatus
atgaagcaccttgcatcttccatcgcattgactctactgttgcctgccgtgcaggcccagcagaccgtatggggccaatgtatgttctggctgtcactggaataagactgtatcaactgctgatatgcttctaggtggcggccaaggctggtctggcccgacgagctgtgttgccggcgcagcctgtagcacactgaatccctgtatgttagatatcgtcctgagtggagacttatactgacttccttagactacgctcagtgtatcccgggagccaccgcgacgtccaccaccctcacgacgacgacggcggcgacgacgacatcccagaccaccaccaaacctaccacgactggtccaactacatccgcacccaccgtgaccgcatccggtaaccctttcagcggctaccagctgtatgccaacccctactactcctccgaggtccatactctggccatgccttctctgcccagctcgctgcagcccaaggctagtgctgttgctgaagtgccctcatttgtttggctgtaagtggccttatcccaatactgagaccaactctctgacagtcgtagcgacgttgccgccaaggtgcccactatgggaacctacctggccgacattcaggccaagaacaaggccggcgccaaccctcctatcgctggtatcttcgtggtctacgacttgccggaccgtgactgcgccgctctggccagtaatggcgagtactcaattgccaacaacggtgtggccaactacaaggcgtacattgacgccatccgtgctcagctggtgaagtactctgacgttcacaccatcctcgtcatcggtaggccgtacacctccgttgcgcgccgcctttctctgacatcttgcagaacccgacagcttggccaacctggtgaccaacctcaacgtcgccaaatgcgccaatgcgcagagcgcctacctggagtgtgtcgactatgctctgaagcagctcaacctgcccaacgtcgccatgtacctcgacgcaggtatgcctcacttcccgcattctgtatcccttccagacactaactcatcaggccatgcgggctggctcggatggcccgccaacttgggccccgccgcaacactcttcgccaaagtctacaccgacgcgggttcccccgcggctgttcgtggcctggccaccaacgtcgccaactacaacgcctggtcgctcagtacctgcccctcctacacccagggagaccccaactgcgacgagaagaagtacatcaacgccatggcgcctcttctcaaggaagccggcttcgatgcccacttcatcatggatacctgtaagtgcttattccaatcgccgatgtgtgccgactaatcaatgtttcagcccggaatggcgtccagcccacgaagcaaaacgcctggggtgactggtgcaacgtcatcggcaccggcttcggtgttcgcccctcgactaacaccggcgatccgctccaggatgcctttgtgtggatcaagcccggtggagagagtgatggcacgtccaactcgacttccccccggtatgacgcgcactgcggatatagtgatgctctgcagcctgctcctgaggctggtacttggttccaggtatgtcatccattagccagatgagggataagtgactgacggacctaggcctactttgagcagcttctgaccaacgctaacccgtccttttaa

SEQ ID NO: 4 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 5 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtttctgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggagcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccaacttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggtttcatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 6 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 7 - DNA, 835 bp, Penicillium sp.
atgctgtcttcgacgactcgcaccctcgcctttacaggccttgcgggccttctgtccgctcccctggtcaaggcccatggctttgtccagggcattgtcatcggtgaccaattgtaagtccctctcttgcagttctgtcgattaactgctggactgcttgcttgactccctgctgactcccaacagctacagcgggtacatcgtcaactcgttcccctacgaatccaacccaccccccgtcatcggctgggccacgaccgccaccgacctgggcttcgtcgacggcacaggataccaaggcccggacatcatctgccaccggaatgcgacgcccgcgccgctgacagcccccgtggccgccggcggcaccgtcgagctgcagtggacgccgtggccggacagccaccacggacccgtcatcacctacctggcgccgtgcaacggcaactgctcgaccgtcgacaagacgacgctggagttcttcaagatcgaccagcagggcctgatcgacgacacgagcccgccgggcacctgggcgtcggacaacctcatcgccaacaacaatagctggaccgtcaccattcccaacagcgtcgcccccggcaactacgtcctgcgccacgagatcatcgccctgcactcggccaacaacaaggacggcgcccagaactacccccagtgcatcaacatcgaggtcacgggcggcggctccgacgcgcctgagggtactctgggcgaggatctctaccatgacaccgacccgggcattctggtcgacatttacgagcccattgcgacgtataccattccggggccgcctgagccgacgttctag

SEQ ID NO: 8 - protein, 253 aa, Penicillium sp.
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 9 - DNA, 1145 bp, Aspergillus fumigatus
atgcgtttctcccttgccgccaccgctcttctcgctggcctggccacggcagcgccttcgagcaacaagaacaacgtcaatcttgataagcttgctcggcgtaatggcatgctttggttcggcactgcagccgatatccctggtacctcagaaacaaccgacaagccttatctgagcatcctgcgcaagcagttcggcgaaatgacacccgcaaacgcattgaaggtgagccagagtgatagtacacctcatctcgtgtcggcgctgaccagacgatgttattcacatagttcatgtataccgagcccgagcagaatgtcttcaacttcactcaaggggactacttcatggacttggccgatcactatggtcacgccgtgcgctgccataacctcgtctgggccagccaagtgtccgactgggtcacctccaggaactggaccgccacagaactcaaagaagtgatgaagaaccacatattcaagaccgtccaacattttggcaagcgctgctacgcgtgggacgtcgtcaatgaagctattaatggggacgggaccttttcctccagtgtgtggtacgacacaattggcgaggaatacttctaccttgcattccagtatgcccaggaagccctggcgcagattcacgccaaccaggtcaagctttactataacgactatggcattgagaaccccggccccaaggcagatgctgttctgaagctagtcgccgagttgcggaagcggggcattcgcattgacggagtcggtctcgagtcccacttcatcgtcggcgagactccttcgctggctgaccagctcgccaccaagaaggcttatatcgaggccggacttgaggtcgccatcaccgaacttgacgtccgcttttctcaggccccgttctacaccgccgaggcccaaaagcagcaggctgccgactactatgctagcgtcgccagttgcaagcatgccggaccgcgctgtgttggtgttgtagtctgggatttcgatgacgcctactcgtggattccgggtaccttcgagggacagggtggcgcctgtctatataatgagacactcgaggtgaagccggccttctatgctgctgccgaggcgttggagaacaagccctgcactgtatgctag

SEQ ID NO: 10 - protein, 364 aa, Aspergillus fumigatus
MRFSLAATALLAGLATAAPSSNKNNVNLDKLARRNGMLWFGTAADIPGTSETTDKPYLSILRKQFGEMTPANALKVSQSDFMYTEPEQNVFNFTQGDYFMDLADHYGHAVRCHNLVWASQVSDWVTSRNWTATELKEVMKNHIFKTVQHFGKRCYAWDVVNEAINGDGTFSSSVWYDTIGEEYFYLAFQYAQEALAQIHANQVKLYYNDYGIENPGPKADAVLKLVAELRKRGIRIDGVGLESHFIVGETPSLADQLATKKAYIEAGLEVAITELDVRFSQAPFYTAEAQKQQAADYYASVASCKHAGPRCVGVVVWDFDDAYSWIPGTFEGQGGACLYNETLEVKPAFYAAAEALENKPCTVC

SEQ ID NO: 11 - DNA, 1400 bp, Aspergillus fumigatus
atggtcgtcctcagcaagctcgtcagcagcattctctttgtctccctggtttcggcgggcgtgatcgacgaacgccaggcagccggcatcaaccaggcgtttacctcccatggcaagaagtactttggcaccgccagtgaccaagctctgctccagaagtcgcagaatgaggccattgtgcgcaaagactttggccagctgacgccggagaatagcatgaagtgggatgcgactgagcgtaggtctctcggccactgtggctgacgttaacttgttgacatgactgtctgtgtagcatcgcaaggaagattcaacttcgctggtgctgatttcctggtatgcaatctgctcatctcggtcgagctcctgctgaaggacaataaataggtcaactatgcaaaacagaatggcaagaaggtccgcggacacaccttaggtattcatgcgccctcacggcatttcgaggatacagccaagctgacagtgtagtctggcactcccaactcccgtcctgggtgtcggctatcagcgacaaaaacaccctgacctcggtgctgaagaaccacatcaccaccgtcatgacccggtacaagggccagatctacgcctgggtattttgccctctatcccacacaatgccagccccagctaatagctgcaaaggacgtcgtcaacgagatcttcaacgaggacggctccctccgcgacagcgtcttctcccgcgtgctgggcgaggactttgtgcggattgccttcgagacggcgcgctctgtggatccctcggcgaagctgtacatcaacgattacaagtaagcttgtggttttgtcgagagatgtactccgtcctggatctgaccatcacagtctcgactcggctagctatggcaaaacccaggggatggtgagatatgtcaagaagtggctggctgcgggcattcctatcgatggaatcggtgagcacaggtcgcggagctgtgtgtgatgattgtacgctgactcttcctgaaggcactcaaacccaccttggtgcgggtgcttcgtccagcgtcaaaggataagtctccttggttttcttgcctacgtaacgctgaccccccgtgtacagcattgactgctcttgcgtcttccggcgtctctgaggtcgccattaccgagctggatatcgcgggtgcgagctcccaggactacgtcaatgtatgtctcctgattgccagtggcagggtcatcgatactaatagaaacaggtcgtcaaggcatgcctggatgtccccaagtgtgtgggaatcaccgtctggggggtgtcggacagggactcgtggcgctccggctcgtctccgctgctgttcgacagcaactaccagcccaaggcggcgtataatgccatcattgctgctctctga

SEQ ID NO: 12 - protein, 323 aa, Aspergillus fumigatus
MVVLSKLVSSILFVSLVSAGVIDERQAAGINQAFTSHGKKYFGTASDQALLQKSQNEAIVRKDFGQLTPENSMKWDATEASQGRFNFAGADFLVNYAKQNGKKVRGHTLWHSQLPSWVSAISDKNTLTSVLKNHITTVMTRYKGQIYAWDVVNEIFNEDGSLRDSVFSRVLGEDFVRIAFETARSVDPSAKLYINDYKLDSASYGKTQGMVRYVKKWLAAGIPIDGIGQTHLGAGASSSVKGALTALASSGVSEVAITELDIAGASSQDYVNVVKACLDVPKCVGITVWGVSDRDSWRSGSSPLLFDSNYQPKAAYNAIIAAL

SEQ ID NO: 13 - DNA, 1415 bp, Aspergillus fumigatus
atggtccatctatcttcattggcagcagccctggctgctctgcctctgtatgtttacccactcacgagaggaggaacagctttgacattgctatagtgtatatggagctggcctgaacacagcagccaaagccaaaggactaaagtactttggttccgccacggacaatccagagctcacggactctgcgtatgtcgcgcaactgagcaacaccgatgattttggtcaaatcacacccggaaactccatgaaggtttgcttacgtctgcctccctggagcattgcctcaaaagctaattggttgttttgtttggatagtgggatgccaccgagccttctcagaattctttttcgttcgcaaatggagacgccgtggtcaatctggcgaacaagaatggccagctgatgcgatgccatactctggtctggcacagtcagctaccgaactggggtatgtaaacgtcttgtctattctcaaatactctctaacagttgacagtctctagcgggtcatggaccaatgcgacccttttggcggccatgaagaatcatatcaccaatgtggttactcactacaaggggaagtgctacgcctgggatgttgtcaatgaaggtttgttgctccatctatcctcaatagttcttttgaaactgacaagcctgtcaatctagccctgaacgaggacggtactttccgtaactctgtcttctaccagatcatcggcccagcatacattcctattgcgttcgccacggctgctgccgcagatcccgacgtgaaactctactacaacgactacaacattgaatactcaggcgccaaagcgactgctgcgcagaatatcgtcaagatgatcaaggcctacggcgcgaagatcgacggcgtcggcctccaggcacactttatcgtcggcagcactccgagtcaatcggatctgacgaccgtcttgaagggctacactgctctcggcgttgaggtggcctataccgaacttgacatccgcatgcagctgccctcgaccgccgcaaagctggcccagcagtccactgacttccaaggcgtggccgcagcatgcgttagcaccactggctgcgtgggtgtcactatctgggactggaccgacaagtactcctgggtccccagcgtgttccaaggctacggcgccccattgccttgggatgagaactatgtgaagaagccagcgtacgatggcctgatggcgggtcttggagcaagcggctccggcaccacaacgaccactactactacttctactacgacaggaggtacggaccctactggagtcgctcagaaatggggacagtgtggcggtattggctggaccgggccaacaacttgtgtcagtggtaccacttgccaaaagctgaatgactggtactcacagtgcctgtaa

SEQ ID NO: 14 - protein, 397 aa, Aspergillus fumigatus
MVHLSSLAAALAALPLVYGAGLNTAAKAKGLKYFGSATDNPELTDSAYVAQLSNTDDFGQITPGNSMKWDATEPSQNSFSFANGDAVVNLANKNGQLMRCHTLVWHSQLPNWVSSGSWTNATLLAAMKNHITNVVTHYKGKCYAWDVVNEALNEDGTFRNSVFYQIIGPAYIPIAFATAAAADPDVKLYYNDYNIEYSGAKATAAQNIVKMIKAYGAKIDGVGLQAHFIVGSTPSQSDLTTVLKGYTALGVEVAYTELDIRMQLPSTAAKLAQQSTDFQGVAAACVSTTGCVGVTIWDWTDKYSWVPSVFQGYGAPLPWDENYVKKPAYDGLMAGLGASGSGTTTTTTTTSTTTGGTDPTGVAQKWGQCGGIGWTGPTTCVSGTTCQKLNDWYSQCL

SEQ ID NO: 15 - DNA, 2376 bp, Aspergillus fumigatus
atggcggttgccaaatctattgctgccgtgctggtagcactgttgcctggtgcgcttgctcaggcgaatacaagctatgttgattacaatgtggaggcgaatccggatctcacccctcagtcggtcgctacgattgacctgtcctttcccgactgcgagaatggaccgctcagcaagactctcgtttgcgacacgtcggctcggccgcatgaccgagctgctgccctggtttccatgttcaccttcgaggagctggtgaacaacacaggcaacactagccctggtgttccaagacttggtctccctccgtaccaagtatggagcgaggctctccatggacttgaccgcgccaacttcacaaacgagggagagtacagctgggccacctcgttccccatgcctatcctgacaatgtcggccttgaaccgaaccctgatcaaccagatcgcgaccatcatcgcaactcaaggacgagctttcaataacgttgggcggtatgggctggacgtgtacgccccgaatataaatgcattcagatcggctatgtggggaagaggtcaagagacccccggagaagacgcttactgcctggcatcggcgtatgcgtacgagtatatcactggcatccagggtggtgttgatccggaacacctcaagttggtggccactgccaaacactatgcgggctacgatcttgagaactgggacggtcactcccgtttgggcaacgatatgaacattacacagcaggaactttccgaatactacacccctcagttccttgttgcagccagagacgccaaagtgcacagtgtcatgtgctcctacaacgcggtaaatggggtgcccagctgcgcaaactcgttcttcctccagaccctcctccgtgacacattcggcttcgtcgaggatggttatgtatccagcgactgcgactcggcgtacaatgtctggaacccgcacgagtttgcggccaacatcacgggggccgctgcagactctatccgggcggggacggacattgattgcggcactacttatcaatactatttcggcgaagcctttgacgagcaagaggtcacccgtgcagaaatcgaaagaggtgtgatccgcctgtacagcaacttggtgcgtctcggctatttcgatggcaatggaagcgtgtatcgggacctgacgtggaatgatgtcgtgaccacggatgcctggaatatctcatacgaagccgctgtagaaggcattgtcctactgaagaacgatggaaccttgcctctcgccaagtcggtccgcagtgttgcattgattgggccctggatgaatgtgacgactcagcttcagggcaactactttggaccggcgccttatctgattagtccgttgaatgccttccagaattctgacttcgacgtgaactacgctttcggcacgaacatttcatcccactccacagatgggttttccgaggcgttgtctgctgcgaagaaatccgacgtcatcatattcgcgggcgggattgacaacactttggaagcagaagccatggatcgcatgaatatcacatggcccggcaatcagctacagctcatcgaccagttgagccaactcggcaaaccgctgatcgtcctccagatgggcggcggccaagtcgactcctcctcgctcaagtccaacaagaatgtcaactccctgatctggggtggataccccggacaatccggcgggcaggctctcctagacatcatcaccggcaagcgcgcccccgccggccgactcgtggtcacgcagtacccggccgaatacgcaacccagttccccgccaccgacatgagcctgcggcctcacggcaataatcccggccagacctacatgtggtacaccggcacccccgtctacgagtttggccacgggctcttctacacgaccttccacgcctccctccctggcaccggcaaggacaagacctccttcaacatccaagacctcctcacgcagccgcatccgggcttcgcaaacgtcgagcaaatgcctttgctcaacttcaccgtgacgatcaccaataccggcaaggtcgcttccgactacactgctatgctcttcgcgaacaccaccgcgggacctgctccatacccgaacaagtggctcgtcggcttcgaccggctggcgagcctggaaccgcacaggtcgcagactatgaccatccccgtgactatcgacagcgtggctcgtacggatgaggccggcaatcgggttctctacccgggaaagtacgagttggccctgaacaatgagcggtcggttgtccttcagtttgtgctgacaggccgagaggctgtgattttcaagtggcctgtagagcagcagcagatttcgtctgcg

SEQ ID NO: 16 - protein, 792 aa, Aspergillus fumigatus
MAVAKSIAAVLVALLPGALAQANTSYVDYNVEANPDLTPQSVATIDLSFPDCENGPLSKTLVCDTSARPHDRAAALVSMFTFEELVNNTGNTSPGVPRLGLPPYQVWSEALHGLDRANFTNEGEYSWATSFPMPILTMSALNRTLINQIATIIATQGRAFNNVGRYGLDVYAPNINAFRSAMWGRGQETPGEDAYCLASAYAYEYITGIQGGVDPEHLKLVATAKHYAGYDLENWDGHSRLGNDMNITQQELSEYYTPQFLVAARDAKVHSVMCSYNAVNGVPSCANSFFLQTLLRDTFGFVEDGYVSSDCDSAYNVWNPHEFAANITGAAADSIRAGTDIDCGTTYQYYFGEAFDEQEVTRAEIERGVIRLYSNLVRLGYFDGNGSVYRDLTWNDVVTTDAWNISYEAAVEGIVLLKNDGTLPLAKSVRSVALIGPWMNVTTQLQGNYFGPAPYLISPLNAFQNSDFDVNYAFGTNISSHSTDGFSEALSAAKKSDVIIFAGGIDNTLEAEAMDRMNITWPGNQLQLIDQLSQLGKPLIVLQMGGGQVDSSSLKSNKNVNSLIWGGYPGQSGGQALLDIITGKRAPAGRLVVTQYPAEYATQFPATDMSLRPHGNNPGQTYMWYTGTPVYEFGHGLFYTTFHASLPGTGKDKTSFNIQDLLTQPHPGFANVEQMPLLNFTVTITNTGKVASDYTAMLFANTTAGPAPYPNKWLVGFDRLASLEPHRSQTMTIPVTIDSVARTDEAGNRVLYPGKYELALNNERSVVLQFVLTGREAVIFKWPVEQQQISSA

SEQ ID NO: 17 - protein, 299 aa, Thielavia terrestris
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNHFDIAMPGGGVGIFNGCSSQFGGLPGAQYGGISSRDQCDSFPAPLKPGCQWRFDWFQNADNPTFTFQQVQCPAEIVARSGCKRNDDSSFPVFTPPSGGNGGTGTPTSTAPGSGQTSPGGGSGCTSQKWAQCGGIGFSGCTTCVSGTTCQKLNDYYSQCL

SEQ ID NO: 18 - protein, 773 aa, Bacillus sp.
AEGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPINDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATNLDPGPDHVWAPEELSLSGEYVRARIKGVNYEPIDRTKYTKVLWDFNDGTKQGFGVNSDSPNKELIAVDNENNTLKVSGLDVSNDVSDGNFWANARLSADGWGKSVDILGAEKLTMDVIVDEPTTVAIAAIPQSSKSGWANPERAVRVNAEDFVQQTDGKYKAGLTITGEDAPNLKNIAFHEEDNNMNNIILFVGTDAADVIYLDNIKVIGTEVEIPVVHDPKGEAVLPSVFEDGTRQGWDWAGESGVKTALTIEEANGSNALSWEFGYPEVKPSDNWATAPRLDFWKSDLVRGENDYVAFDFYLDPVRATEGAMNINLVFQPPTNGYWVQAPKTYTINFDELEEANQVNGLYHYEVKINVRDITNIQDDTLLRNMMIIFADVESDFAGRVFVDNVRFEGAATTEPVEPEPVDPGEETPPVDEKEAKKEQKEAEKEEKEE

(19) United States
(12) Patent Application Publication
(10) Pub. No.: US 2018/0171271 A1
(43) Pub. Date: Jun. 21, 2018

(54) LAUNDRY DETERGENT COMPOSITION, METHOD FOR WASHING AND USE OF COMPOSITION

(71) Applicant: NOVOZYMES A/S, Bagsvaerd (DK)

(72) Inventors: Jan Peter Skagerlind, Helsingborg (SE); Paulo Cesar Barjona, Parana (BR)

(73) Assignee: NOVOZYMES A/S, Bagsvaerd (DK)

(21) Appl. No.: 15/580,586

(22) PCT Filed: Jun. 23, 2016
(86) PCT No.: PCT/EP2016/064526

§ 371 Date: Dec. 7, 2017

(30) Foreign Application Priority Data
Jun. 30, 2015 (EP) 15174596.5

(51) Int. Cl. C11D 3/386; C11D 3/00; C11D 11/00; C12N 9/42; C07K 14/385

(52) U.S. Cl. CPC C11D 3/38636; C11D 3/38645; C11D 3/001; C11D 11/0017; C07K 14/385; C12Y 302/01004; C12Y 302/01021; C12Y 302/01091; C12N 9/2445

(57) ABSTRACT

The patent application discloses the use of enzymes capable of degrading cellulosic material for removing and preventing build up of fuzz and pills and for improving the whiteness of the enzyme. Further a method for washing is disclosed.

### FIELD OF THE INVENTION

The present invention concerns a laundry detergent composition comprising an enzyme capable for degrading cellulosic material. The invention further concerns a washing method, the use of enzymes capable for degrading cellulosic material for washing and a method for cleaning washing machines.

### REFERENCE TO A SEQUENCE LISTING

This application contains a Sequence Listing in computer readable form. The computer readable form is incorporated herein by reference.

### BACKGROUND OF THE INVENTION

Use of enzymes in laundry detergents is well known. Also use of enzymes capable for degrading cellulosic material is known for laundry purpose. However, cellulose degrading enzymes for laundry should be selected carefully as laundry textile serve as substrate for the enzymes.

The degradation of cellulosic material in washing machines is often a challenge. Cellulosic fibers may be cleaved from textile during wash with enzymes capable for degrading cellulosic material and tend to clog filters, pipes and drains in washing machines. The drains and filters thus need to be cleaned manually from time to time.

Wearing, washing and tumble drying of fabric and textile exposes the textile to mechanical stress which damages the textile and fabric by breaking the fibers in the fabric/textile and thereby causing the textile/fabric to be covered with fuzz and pills. This gives the fabric or textile a worn look.

### SUMMARY OF THE INVENTION

The present invention concerns use of a laundry composition comprising one or more enzymes capable of degrading cellulosic material, a method for laundering a textile comprising contacting the textile to one or more enzymes capable of degrading cellulosic material and a cleaning method for cleaning the interior of an automated washing machine.

### Definitions

Anti-pilling: The term “anti-pilling” denotes removal of pills from the textile surface and/or prevention of formation of pills on the textile surface.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20 (polyoxyethylene sorbitan monolaurate).

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. For purposes of the present invention, one unit of beta-xylosidase is defined as 1.0 μmole of p-nitrophenolate anion produced per minute at 40° C., pH 5 from 1 mM p-nitrophenyl-beta-D-xyloside as substrate in 100 mM sodium citrate containing 0.01% TWEEN® 20.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teeri, 1997, Crystalline cellulose degradation: New insight into the function of cellobiohydrolases, Trends in Biotechnology 15: 160-167; Teeri et al., 1998, Trichoderma reesei cellobiohydrolases: why so efficient on crystalline cellulose?, Biochem. Soc. Trans. 26: 173-178). Cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581. In the present invention, the Tomme et al. method can be used to determine cellobiohydrolase activity.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No1 filter paper, microcrystalline cellulose, bacterial cellulose, algal cellulose, cotton, pretreated lignocellulose, etc. The most common total cellulolytic activity assay is the filter paper assay using Whatman No1 filter paper as the substrate. The assay was established by the International Union of Pure and Applied Chemistry (IUPAC) (Ghose, 1987, Measurement of cellulase activities, Pure Appl. Chem. 59: 257-68).

For purposes of the present invention, cellulolytic enzyme activity is determined by measuring the increase in hydrolysis of a cellulosic material by cellulolytic enzyme(s) under the following conditions: 1-50 mg of cellulolytic enzyme protein/g of cellulose in PCS (or other pretreated cellulosic material) for 3-7 days at a suitable temperature, e.g., 50° C., 55° C., or 60° C., compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in vegetable food products, such as salad, tomatoes, spinach, cabbage, grain or the like.

Detergent component: The term “detergent component” is defined herein to mean the types of chemicals which can be used in detergent compositions for laundry. Examples of detergent components are surfactants, builders, chelators or chelating agents, bleach system or bleach components, polymers, fabric conditioners, foam boosters, suds suppressors, dyes, perfume, tannish inhibitors, bactericides, fungicides, soil suspending agents, anti-corrosion agents, enzyme inhibitors or stabilizers, enzyme activators, transferase(s), hydrolytic enzymes, oxido reductases, blueing agents and fluorescent dyes, antioxidants, and solubilizers.

Detergent Composition: The term “detergent composition” refers to compositions that find use in the removal of undesired compounds from surfaces to be cleaned, such as textile surfaces. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). The detergent composition may contain one or more enzymes such as hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, beta-glucanases, arabinosidases, hyaluronidase, chondroitinase, laccase, DNase, chlorophyllases, amylases, perhydrolases, peroxidases, xanthanase and mixtures thereof. The detergent composition may further comprise detergent component such as surfactants, builders, chelators or chelating agents, bleach system or bleach components, polymers, fabric conditioners, foam boosters, suds suppressors, dyes, perfume, tannish inhibitors, bactericides, fungicides, soil suspending agents, anti-corrosion agents, enzyme inhibitors or stabilizers, enzyme activators, transferase(s), hydrolytic enzymes, oxido reductases, blueing agents and fluorescent dyes, antioxidants, and solubilizers.

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Fabric softener: A Fabric softener (also called fabric conditioner) is a composition that is typically applied to laundry during the rinse cycle in a washing machine or when washing by hand. Fabric softeners are available as solutions and solids, and may also be impregnated in dryer sheets used in a clothes dryer.

Fabric softener agent: A fabric softener agent is an ingredient that is comprised in fabric softener compositions such as chemical compounds that are electrically charged. These compounds causes threads in the fabric to lift up from the surface of the textile and thereby gives the fabric a softer feel of the textile. In one embodiment the fabric softener agent is one ore more cationic softeners. The cationic softeners bind by electrostatic attraction to the negatively charged groups on the surface of the textile and neutralize their charge and thereby impart lubricity.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Fragment: The term “fragment” means a polypeptide having one or more (e.g., several) amino acids absent from the amino and/or carboxyl terminus of a mature polypeptide main; wherein the fragment has enzyme activity. In one aspect, a fragment contains at least 85%, e.g., at least 90% or at least 95% of the amino acid residues of the mature polypeptide of an enzyme.

Hemicellulolytic enzyme or hemicellulase: The term “hemicellulolytic enzyme” or “hemicellulase” means one or more (e.g., several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature, e.g., 50° C., 55° C., or 60° C., and pH, e.g., 5.0 or 5.5.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Improved wash performance: The term “improved wash performance” is defined herein as a laundry detergent composition displaying an increased wash performance relative to the wash performance of a laundry detergent composition without the inventive enzyme preparation, e.g. by increased soil removal.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Polypeptide having cellulolytic enhancing activity: The term “polypeptide having cellulolytic enhancing activity” means a GH61 polypeptide that catalyzes the enhancement of the hydrolysis of a cellulosic material by enzyme having cellulolytic activity. For purposes of the present invention, cellulolytic enhancing activity is determined by measuring the increase in reducing sugars or the increase of the total of cellobiose and glucose from the hydrolysis of a cellulosic material by cellulolytic enzyme under the following conditions: 1-50 mg of total protein/g of cellulose in PCS, wherein total protein is comprised of 50-99.5% w/w cellulolytic enzyme protein and 0.5-50% w/w protein of a GH61 polypeptide having cellulolytic enhancing activity for 1-7 days at a suitable temperature, e.g., 50° C., 55° C., or 60° C., and pH, e.g., 5.0 or 5.5, compared to a control hydrolysis with equal total protein loading without cellulolytic enhancing activity (1-50 mg of cellulolytic protein/g of cellulose in PCS). In a preferred aspect, a mixture of CELLUCLAST® 1.5L (Novozymes NS, Bagsværd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The GH61 polypeptides having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Pretreated corn stover: The term “PCS” or “Pretreated Corn Stover” means a cellulosic material derived from corn stover by treatment with heat and dilute sulfuric acid, alkaline pretreatment, or neutral pretreatment.

Remission value: Wash performance is expressed as a Remission value of the stained swatches. After washing and rinsing the swatches were spread out flat and allowed to air dry at room temperature overnight. All washes swatches are evaluated the day after the wash. Light reflectance evaluations of the swatches were done using a Macbeth Color Eye 7000 reflectance spectrophotometer with very small aperture. The measurements were made without UV in the incident light and remission at 460 nm was extracted.

Rinse cycle: The term “rinse cycle” is defined herein as a rinsing operation wherein textile is exposed to water for a period of time by circulating the water and optionally mechanically treat the textile in order to rinse the textile and finally the superfluous water is removed. A rinse cycle may be repeated one, two, three, four, five or even six times at the same or at different temperatures.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having enzyme activity. In one aspect, a subsequence contains at least 85%, e.g., at least 90% or at least 95% of the nucleotides of the mature polypeptide coding sequence of an enzyme.

Variant: The term “variant” means a polypeptide having enzyme activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Wash cycle: The term “wash cycle” is defined herein as a washing operation wherein textile is exposed to the wash liquor for a period of time by circulating the wash liquor and optionally mechanically treat the textile in order to clean the textile and finally the superfluous wash liquor is removed. A wash cycle may be repeated one, two, three, four, five or even six times at the same or at different temperatures. Hereafter the textile is generally rinsed and dried. One of the wash cycles can be a soaking step, where the textile is left soaking in the wash liquor for a period.

Wash liquor: The term “wash liquor” is intended to mean the solution or mixture of water and a detergent component optionally including enzymes used for laundry.

Wash time: The term “wash time” is defined herein as the time it takes for the entire washing process; i.e. the time for the wash cycle(s) and rinse cycle(s) together.

Whiteness: The term “Whiteness” is defined herein as a broad term with different meanings in different regions and for different consumers. Loss of whiteness can e.g. be due to greying, yellowing, or removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum etc.); redeposition (greying, yellowing or other discolourations of the object) (removed soils reassociate with other parts of textile, soiled or unsoiled); chemical changes in textile during application; and clarification or brightening of colours.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the processes of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, Recent progress in the assays of xylanolytic enzymes, 2006, Journal of the Science of Food and Agriculture 86(11): 1636-1647; Spanikova and Biely, 2006, Glucuronoyl esterase—Novel carbohydrate esterase produced by Schizophyllum commune, FEBS Letters 580(19): 4597-4601; Herrmann, Vrsanska, Jurickova, Hirsch, Biely, and Kubicek, 1997, The beta-D-xylosidase of Trichoderma reesei is a multifunctional beta-D-xylan xylohydrolase, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. The most common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 (4-(1,1,3,3-tetramethylbutyl)phenyl-polyethylene glycol) and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

For purposes of the present invention, xylan degrading activity is determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, A new reaction for colorimetric determination of carbohydrates, Anal. Biochem 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

### DETAILED DESCRIPTION OF THE INVENTION

The present invention concerns the use of a laundry composition for use in an laundry processes. The composition comprises one or more enzymes capable of degrading cellulosic material. The composition can be a laundry detergent composition or a fabric softener composition.

The enzymes capable of degrading cellulosic material can be selected from the group consisting of Aspergillus fumigatus GH10 xylanases, Aspergillus fumigatus beta-xylosidases, Aspergillus fumigatus cellobiohydrolase I, Aspergillus fumigatus cellobiohydrolase II, Aspergillus fumigatus beta-glucosidase variants and Penicillium sp. (emersonii) GH61 polypeptide.

The inventor has found that these enzymes are superior in degrading cellulosic material released from laundry items during a laundry process, where the enzymes of the invention, in addition to the removal of fuzz and pills from cotton textile, improving whiteness and color clarification, is capable of degrading the cellulosic material to an extend so that the cellulosic material do not clog filters, pipes and drain of the laundry washing machine. Therefore the consumer needs not to clean manually the filter or drain of the laundry washing machine as often as usual. Further, the washed textile appears without fuzz and pills without losing too much textile strength as demonstrated in example 2.

The inventor has found that use of the laundry composition of the invention should comprise a surfactant or a fabric softener agent and an enzyme preparation comprising one or more enzymes capable of degrading cellulosic material, wherein the one or more enzymes capable of degrading cellulosic material comprises:

(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof.

The Aspergillus fumigatus cellobiohydrolase I or homolog thereof of the enzyme preparation is selected from the group consisting of:
(i) a cellobiohydrolase I comprising or consisting of the mature polypeptide of SEQ ID NO: 2;
(ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2;
(iii) a cellobiohydrolase I encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1; and
(iv) a cellobiohydrolase I encoded by a polynucleotide that hybridizes under at least high stringency conditions, very high stringency conditions, with the mature polypeptide coding sequence of SEQ ID NO: 1 or the full-length complement thereof.

The Aspergillus fumigatus cellobiohydrolase II or homolog thereof is selected from the group consisting of:
(i) a cellobiohydrolase II comprising or consisting of the mature polypeptide of SEQ ID NO: 4;
(ii) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4;
(iii) a cellobiohydrolase II encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 3; and
(iv) a cellobiohydrolase II encoded by a polynucleotide that hybridizes under at least high stringency conditions, very high stringency conditions, with the mature polypeptide coding sequence of SEQ ID NO: 3 or the full-length complement thereof.

The Aspergillus fumigatus beta-glucosidase or homolog thereof is selected from the group consisting of:
(i) a beta-glucosidase comprising or consisting of the mature polypeptide of SEQ ID NO: 6;
(ii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6;
(iii) a beta-glucosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5;
(iv) a beta-glucosidase encoded by a polynucleotide that hybridizes under at least high stringency conditions, very high stringency conditions, with the mature polypeptide coding sequence of SEQ ID NO: 5 or the full-length complement thereof; and
(v) a beta-glucosidase variant comprising a substitution at one or more positions corresponding to positions 100, 283, 456, and 512 of the mature polypeptide of SEQ ID NO: 6, wherein the variant has beta-glucosidase activity; and

The Penicillium sp. GH61 polypeptide having cellulolytic enhancing activity or homolog thereof is selected from the group consisting of:
(i) a GH61 polypeptide having cellulolytic enhancing activity comprising or consisting of the mature polypeptide of SEQ ID NO: 6;
(ii) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
(iii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
(iv) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.

The beta-glucosidase variant of the enzyme preparation comprises one or more (several) substitutions selected from the group consisting of G142S, Q183R, H266Q, and D703G.

In an embodiment of the invention the enzyme preparation further comprises Penicillium emersonii GH61A polypeptide having cellulolytic enhancing activity disclosed in WO 2011/041397 and Aspergillus fumigatus beta-glucosidase (SEQ ID NO: 2 of WO 2005/047499) or SEQ ID NO: 6 herein or a variant thereof with the following substitutions F100D, S283G, N456E, F512Y.

In a preferred embodiment the invention the enzyme preparation comprises:
i. an Aspergillus fumigatus cellobiohydrolase I preferably the one shown in SEQ ID NO: 2 herein;
ii. an Aspergillus fumigatus cellobiohydrolase II preferably the one shown in SEQ ID NO: 4 herein;
iii. an Aspergillus fumigatus beta-glucosidase preferably the one shown in SEQ ID NO: 6 herein; or variant thereof, such as one with the following substitutions: F100D, S283G, N456E, F512Y (using SEQ ID NO: 6 herein for numbering); and
iv. a Penicillium sp. GH61 polypeptide having cellulolytic enhancing activity preferably the one shown in SEQ ID NO: 8 herein; or homologs thereof.

The enzyme preparation can further comprise one or more enzymes selected from the group consisting of:

an Aspergillus fumigatus xylanase or homolog thereof,

an Aspergillus fumigatus beta-xylosidase or homolog thereof; or

a combination of (i) and (ii).

The Aspergillus fumigatus xylanase or homolog thereof is selected from the group consisting of:

an Aspergillus fumigatus xylanase comprising or consisting of the mature polypeptide of SEQ ID NO: 10, SEQ ID NO: 12, or SEQ ID NO: 14;

a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10, SEQ ID NO: 12, or SEQ ID NO: 14;

a xylanase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 9, SEQ ID NO: 11, or SEQ ID NO: 13; and

a xylanase encoded by a polynucleotide that hybridizes under at least high stringency conditions, very high stringency conditions, with the mature polypeptide coding sequence of SEQ ID NO: 9, SEQ ID NO: 11, or SEQ ID NO: 13; or the full-length complement thereof.

The Aspergillus fumigatus beta-xylosidase or homolog thereof is selected from the group consisting of:

beta-xylosidase comprising or consisting of the mature polypeptide of SEQ ID NO: 16;

a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;

a beta-xylosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 15; and

a beta-xylosidase encoded by a polynucleotide that hybridizes under at least high stringency conditions, very high stringency conditions, with the mature polypeptide coding sequence of SEQ ID NO: 15 or the full-length complement thereof.

The enzymes capable of degrading cellulosic material can be present in a laundry detergent composition comprising at least one more enzyme. The additional enzyme can be selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, R-glucanases, arabinosidases, hyaluronidase, chondroitinase, laccase, DNase chlorophyllases, amylases, perhydrolases, peroxidases, xanthanase and mixtures thereof. In one embodiment of the invention the at least one more enzyme is selected from proteases, lipases, mannanases, pectate lyases and amylases.

The laundry detergent composition comprises a surfactant, which can be selected from the group consisting of anionic, cationic, non-ionic, semi-polar and zwitterionic surfactants. In addition other detergent components such as builders and polymers can be comprised in the laundry detergent composition.
1. The invention further concerns a method for laundering a textile comprising the steps of:
   a) Contacting the textile with a wash liquor comprising laundry detergent composition;
   b) Completing at least one wash cycle;
   c) contacting the textile with water comprising a fabric softener composition; and
   d) completing at least one rinse cycle;

wherein the detergent composition and/or the fabric softener composition comprise

an enzyme preparation comprising one or more enzymes capable of degrading cellulosic material and wherein the one or more enzymes capable of degrading cellulosic material comprises:
i. an Aspergillus fumigatus cellobiohydrolase I;
ii. an Aspergillus fumigatus cellobiohydrolase II;
iii. an Aspergillus fumigatus beta-glucosidase or variant thereof; and
iv. a Penicillium sp. GH61 polypeptide having cellulolytic enhancing activity; or homologs thereof; and

wherein the method prevents build-up and/or removal of fuzz and pills from a textile and improves the whiteness of the textile.

In order to facilitate the degradation of the cellulosic material, an aqueous solution of an acidic material can be used during the washing process. The acidic material should be capable of lowering the pH to below 5. It is believed that when an acidic solution is used on cellulosic material, the structure of the cellulosic material opens up and is more susceptible to the enzymes capable of degrading cellulosic material. The process of degrading the cellulosic material is thereby faster than the degradation process without use of acid.

The exposing of the acidic material can be done a separate step in the laundering process, where the textile is exposed to an aqueous solution of an acidic material before the textile is exposed to washing liquor. This step can be performed in several ways. One way is by simply adding an acidic material to the interior of the laundry washing machine before starting the washing process. The acidic material will then dissolve when contacted with water. Another way is by circulating an aqueous solution of the acidic material before exposing the textile to the wash liquor, e.g. in a pre-wash step. The acidic solution can be exposed to the textile during a rinsing step. In one embodiment the fabric softener used during rinsing has a pH below 5. The washing of textile can be carried out by hand or with a laundry washing machine.

Alternatively, the laundry detergent composition is a powder or a granule and the acidic material is the outer layer of the powder granules. The acidic material is thereby released from the laundry detergent composition and dissolved in the water in the washing machine before the laundry detergent composition is released. Another option is that the acidic material and the laundry detergent composition are contained in a pouch having two or more compartments, where the acidic material is contained in one compartment and the laundry detergent composition is contained in the other compartment. The compartment with the acidic material can then be released and dissolved before the laundry detergent composition is released from the other compartment. Further, the composition can be a tablet having two or more layers, wherein the acidic material is the outer layer of the bar, which will then be released as described above.

The acidic solution should be capable of lowering the pH below 4 during at least a period of the washing cycle. The pH may be even further lowered e.g. to below pH 3.5, such as below pH 3, below pH 2.5 or below pH 2. The period of lowering the pH may be at least 1 minute, such as at least 2 minutes, at least 3 minutes, at least 4 minutes, at least 5 minutes, at least 6 minutes or at least 7 minutes.

The ability of lowering the pH during the rinsing step is due to a buffering agent selected from the group consisting of citric acid, acetic acid, potassium dihydrogen phosphate, boric acid, diethyl barbituric acid, Carmody buffer and Britton-Robinson buffer.

The laundry detergent composition may be in the form of a powder, a bar, a homogenous tablet, a tablet having two or more layers, a pouch having one or more compartments, a regular or compact powder, a granule, a paste, a gel, or a regular, compact or concentrated liquid. The composition can be a powder or a granule where the acidic material is coated on the powder or granule as an outer layer. Alternatively the composition is a tablet having two or more layers, wherein the acidic material is the outer layer of the bar.

The composition can be a pouch having at least two compartments, wherein the acidic material is present in one compartment and is released before content of the other compartment(s).

The inventor has found that the enzyme preparation of the invention is very good at degrading cellulosic material. When the enzyme preparation is used in a laundering process as described above in combination with an aqueous solution of an acidic material, the degradation of cellulosic material from fuzz and pills can be even better than degradation of cellulosic material in a similar process without the use of an acidic solution. The enzyme preparation comprises: Aspergillus fumigatus GH10 xylanase, Aspergillus fumigatus beta-xylosidase, Aspergillus fumigatus cellobiohydrolase I, Aspergillus fumigatus cellobiohydrolase II, Aspergillus fumigatus beta-glucosidase variant and Penicillium sp. (emersonii) GH61 polypeptide.

The one or more enzymes capable of degrading cellulosic material which is used in the laundry washing process can be comprised in a laundry detergent composition according to the present invention.

The one or more enzymes capable of degrading cellulosic material can be used for degrading cellulosic material during a laundry process. An acidic material may be used during the laundry process. The one or more enzymes capable of degrading cellulosic material can also be used for cleaning the interior of a laundry washing machine, e.g. cleaning of the drain for surplus of cellulose from fuzz and pills, where the cellulosic material remains after a washing process.

The invention further concerns a method for cleaning the interior of a laundry washing machine, which method comprises exposing the interior of the laundry washing machine to one or more enzymes capable of degrading cellulosic material.

The one or more enzymes capable of degrading cellulosic material can be an enzyme preparation comprising:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof, and which enzymes are described in details above.

### Concentration of the Enzyme

In one embodiment of the present invention, the polypeptide of the present invention may be used in the laundry detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The enzyme(s) of the detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

A polypeptide of the present invention may also be incorporated in the detergent formulations disclosed in WO97/07202, which is hereby incorporated by reference.

### Surfactants

The laundry detergent composition may comprise one or more surfactants, which may be anionic and/or cationic and/or non-ionic and/or semi-polar and/or zwitterionic, or a mixture thereof.

In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein, the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein, the detergent will usually contain from about from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein, the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein, the detergent will usually contain from about 0% to about 20% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein, the detergent will usually contain from about 0% to about 20% by weight of a zwitterionic surfactant. Non-limiting examples of zwitterionic surfactants include betaines such as alkyldimethylbetaines, sulfobetaines, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a washing detergent, the level of builder is typically 40-65%, particularly 50-65%. The builder and/or co-builder may particularly be a chelating agent that forms water-soluble complexes with Ca and Mg. Any builder and/or co-builder known in the art for use in laundry cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-01), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (H EDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The detergent may contain 0-30% by weight, such as about 1% to about 20%, of a bleaching system. Any bleaching system known in the art for use in laundry cleaning detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

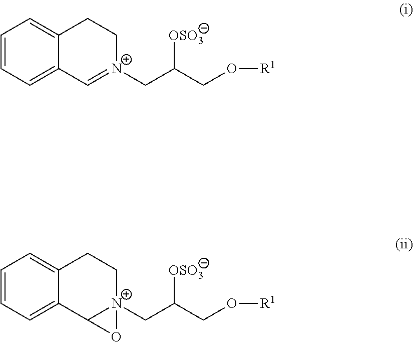

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) pre-formed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Enzymes

The detergent additive as well as the detergent composition may comprise one or more [additional] enzymes such as a protease, lipase, cutinase, an amylase, carbohydrase, cellulase, pectinase, mannanase, arabinase, galactanase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes NS) Carezyme Premium™ (Novozymes NS), Celluclean™ (Novozymes NS), Celluclean Classic™ (Novozymes NS), Cellusoft™ (Novozymes NS), Whitezyme™ (Novozymes NS), Clazinase™, and Puradax HA™ (Genencor International Inc.), and KAC-500(B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes NS).

### Cellulase

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Suitable cellulases include a fungal cellulase from Humicola insolens (U.S. Pat. No. 4,435,307) or from Trichoderma, e.g. T. reesei or T. viride. Examples of cellulases are described in EP 0 495 257. Other suitable cellulases are from Thielavia e.g. Thielavia terrestris as described in WO 96/29397 or Fusarium oxysporum as described in WO 91/17244 or from Bacillus as described in, WO 02/099091 and JP 2000210081. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 Commercially available cellulases include Carezyme®, Celluzyme®, Celluclean®, Celluclast® and Endolase®; Renozyme®; Whitezyme® (Novozymes NS) Puradax®, Puradax HA, and Puradax EG (available from Genencor).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes NS).

### Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V41, S9R, A15T, K27R, *36D, V68A, N76D, N87S,R, *97E, A98S, S99G,D,A, S99AD, S101G,M,R S103A, V1041,Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes NS), those sold under the tradename Maxatase®, Maxacal®, Maxapem®, Purafect®, Purafect Prime®, Purafect MAO, Purafect Ox®, Purafect OxP®, Puramax®, Properase®, FN2®, FN3®, FN4®, Excellase®, Eraser®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes NS), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

Suitable amylases which can be used together with the enzyme preparation of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N1280+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+1203Y+G476K

E187P+1203Y+R458N+T459S+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, 1181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I

wherein the variants optionally further comprises a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™ Stainzyme Plus™, Natalase™, Liquozyme X and BAN™ (from Novozymes NS), and Rapidase™, Purastar™/Effectenz™, Powerase, Preferenz S1000, Preferenz S100 and Preferenz S110 (from Genencor International Inc./DuPont).

### Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In a preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are polyethyleneglycol (PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry cleaning detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry cleaning detergents may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diary) pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The enzyme preparation of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, enzyme preparation, optionally one or more additional enzymes, a protease inhibitor, and a salt of a monovalent cation and an organic anion may be prepared and and the mixture is then plodded. The enzyme preparation and optional additional enzymes may be added at the same time as the protease inhibitor for example in liquid form. Besides the mixing step and the plodding step, the process may further comprise the steps of milling, extruding, cutting, stamping, cooling and/or wrapping.

### Formulation of Enzyme in Co-Granule

The enzyme of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component.

WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise an enzyme of the invention and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

### The Invention is Further Summarized in the Following Paragraphs:

1. Use of a laundry detergent composition comprising an enzyme preparation comprising one or more enzymes capable of degrading cellulosic material and a surfactant or a fabric softener agent, wherein the one or more enzymes capable of degrading cellulosic material comprises:
   (i) an Aspergillus fumigatus cellobiohydrolase I;
   (ii) an Aspergillus fumigatus cellobiohydrolase II;
   (iii) an Aspergillus fumigatus beta-glucosidase or variant thereof; and
   (iv) a Penicillium sp. GH61 polypeptide having cellulolytic enhancing activity; or homologs thereof;
      and wherein the composition is used for preventing build-up and/or removal of fuzz and pills from a textile and for improving the whiteness of the textile by exposing the textile to the composition during a laundry process.
2. Use according to paragraph 1, wherein the Aspergillus fumigatus cellobiohydrolase I or homolog thereof of the enzyme preparation is selected from the group consisting of:
   (i) a cellobiohydrolase I comprising or consisting of the mature polypeptide of SEQ ID NO: 2;
   (ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2;
   (iii) a cellobiohydrolase I encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1; and
   (iv) a cellobiohydrolase I encoded by a polynucleotide that hybridizes under at least high stringency conditions, very high stringency conditions, with the mature polypeptide coding sequence of SEQ ID NO: 1 or the full-length complement thereof;
wherein the Aspergillus fumigatus cellobiohydrolase II or homolog thereof is selected from the group consisting of:
   (i) a cellobiohydrolase II comprising or consisting of the mature polypeptide of SEQ ID NO: 4;
   (ii) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4;
   (iii) a cellobiohydrolase II encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 3; and
   (iv) a cellobiohydrolase II encoded by a polynucleotide that hybridizes under at least high stringency conditions, very high stringency conditions, with the mature polypeptide coding sequence of SEQ ID NO: 3 or the full-length complement thereof;
wherein the Aspergillus fumigatus beta-glucosidase or homolog thereof is selected from the group consisting of:
   (i) a beta-glucosidase comprising or consisting of the mature polypeptide of SEQ ID NO: 6;
   (ii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6;
   (iii) a beta-glucosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5;
   (iv) a beta-glucosidase encoded by a polynucleotide that hybridizes under at least high stringency conditions, very high stringency conditions, with the mature polypeptide coding sequence of SEQ ID NO: 5 or the full-length complement thereof; and
   (v) a beta-glucosidase variant comprising a substitution at one or more positions corresponding to positions 100, 283, 456, and 512 of the mature polypeptide of SEQ ID NO: 6, wherein the variant has beta-glucosidase activity; and
wherein the Penicillium sp. GH61 polypeptide having cellulolytic enhancing activity or homolog thereof is selected from the group consisting of:
   (i) a GH61 polypeptide having cellulolytic enhancing activity comprising or consisting of the mature polypeptide of SEQ ID NO: 8;
   (ii) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
   (iii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
   (iv) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.
3. Use according to any of the preceding paragraphs, wherein the beta-glucosidase variant of the enzyme preparation comprises one or more (several) substitutions selected from the group consisting of G142S, Q183R, H266Q, and D703G.
4. Use according to any of the preceding paragraphs, wherein the enzyme preparation further comprises one or more enzymes selected from the group consisting of:
   (i) an Aspergillus fumigatus xylanase or homolog thereof,
   (ii) an Aspergillus fumigatus beta-xylosidase or homolog thereof; or
   (iii) a combination of (i) and (ii);
wherein the Aspergillus fumigatus xylanase or homolog thereof is selected from the group consisting of:
   (i) an Aspergillus fumigatus xylanase comprising or consisting of the mature polypeptide of SEQ ID NO: 10, SEQ ID NO: 12, or SEQ ID NO: 14;
   (ii) a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10, SEQ ID NO: 12, or SEQ ID NO: 14;
   (iii) a xylanase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 9, SEQ ID NO: 11, or SEQ ID NO: 13; and
   (iv) a xylanase encoded by a polynucleotide that hybridizes under at least high stringency conditions, very high stringency conditions, with the mature polypeptide coding sequence of SEQ ID NO: 9, SEQ ID NO: 11, or SEQ ID NO: 13; or the full-length complement thereof; and
wherein the Aspergillus fumigatus beta-xylosidase or homolog thereof is selected from the group consisting of:
   (i) beta-xylosidase comprising or consisting of the mature polypeptide of SEQ ID NO: 16;
   (ii) a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;
   (iii) a beta-xylosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 15; and
   (iv) a beta-xylosidase encoded by a polynucleotide that hybridizes under at least high stringency conditions, very high stringency conditions, with the mature polypeptide coding sequence of SEQ ID NO: 15 or the full-length complement thereof.
5. Use according to any of the preceding paragraphs, wherein the composition comprises at least one enzyme in addition to the enzymes in the enzyme preparation.
6. Use according to any of the preceding paragraphs, wherein the at least one more enzyme is selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, R-glucanases, arabinosidases, hyaluronidase, chondroitinase, laccase, DNase chlorophyllases, amylases, perhydrolases, peroxidases, xanthanase and mixtures thereof.
7. Use according to paragraph 6, wherein the at least one more enzyme is selected from the group consisting of proteases, lipases, mannanases, pectate lyases, amylases or mixtures thereof.
8. Use according to any of the preceding paragraphs, wherein the surfactant is selected from the group consisting of anionic, cationic, non-ionic, semi-polar and zwitterionic surfactants.
9. Use according to any of the preceding paragraphs, wherein the composition further comprises one or more builders and one or more polymers.
10. Use according to any of the preceding composition paragraphs, wherein the composition further comprises one or more components selected from the group consisting of polymers, bleaching systems, bleach activators, bleach catalysts, silicates and dyestuff.
11. Use according to any of the preceding paragraphs, wherein the composition further comprises an acidic material.
12. Use according to any of the preceding paragraphs, wherein the acidic material is selected from the group consisting of citric acid, acetic acid, potassium dihydrogen phosphate, boric acid, diethyl barbituric acid, Carmody buffer and Britton-Robinson buffer
13. Use according to any of the preceding paragraphs, wherein the composition is in the form of a powder, a bar, a homogenous tablet, a tablet having two or more layers, a pouch having one or more compartments, a regular or compact powder, a granule, a paste, a gel, or a regular, compact or concentrated liquid.
14. Use according to any of the preceding paragraphs, wherein the composition is a powder or a granule and the acidic material is the outer layer of the powder granules.
15. Use according to any of the preceding paragraphs, wherein the composition is a tablet having two or more layers, wherein the acidic material is the outer layer of the bar.
16. Use according to any of the preceding paragraphs, wherein the composition is a pouch having at least two compartments, wherein the acidic material is present in one compartment and is released before content of the other compartment(s).
17. Use according to any of the preceding paragraphs, wherein the composition is for used in house hold laundry or industrial laundry.
18. A method for laundering a textile comprising the steps of:
   e) Contacting the textile with a wash liquor comprising laundry detergent composition;
   f) Completing at least one wash cycle;
   g) contacting the textile with water comprising a fabric softener composition; and
   h) completing at least one rinse cycle;
wherein the detergent composition and/or the fabric softener composition comprise an enzyme preparation comprising one or more enzymes capable of degrading cellulosic material and wherein the one or more enzymes capable of degrading cellulosic material comprises:
   v. an Aspergillus fumigatus cellobiohydrolase I;
   vi. an Aspergillus fumigatus cellobiohydrolase II;
   vii. an Aspergillus fumigatus beta-glucosidase or variant thereof; and
   viii. a Penicillium sp. GH61 polypeptide having cellulolytic enhancing activity; or homologs thereof; and
      wherein the method prevents build-up and/or removal of fuzz and pills from a textile and improves the whiteness of the textile.
19. Method according to paragraph 18, wherein the method further comprises exposing the textile to an acidic solution.
20. Method according to paragraph 19, wherein the acidic solution having a pH below 5 is exposed to the textile during a pre-wash or during rinsing.
21. Method according to any of the preceding method paragraphs, wherein the laundry method is carried out by hand or machine.
22. Method according to any of paragraphs 18-22, wherein the concentration of the enzyme preparation is in the range of 0.03 to 5 gram/liter wash liquor.
23. Use of an enzyme preparation for cleaning, wherein the enzyme preparation comprises one or more enzymes capable of degrading cellulosic material, wherein enzyme preparation comprises:
   (i) an Aspergillus fumigatus cellobiohydrolase I;
   (ii) an Aspergillus fumigatus cellobiohydrolase II;
   (iii) an Aspergillus fumigatus beta-glucosidase or variant thereof; and
   (iv) a Penicillium sp. GH61 polypeptide having cellulolytic enhancing activity; or homologs thereof.
24. Use according to paragraph 23 for laundering of textile or cleaning of the interior of a washing machine such as walls, nozzles, pumps, sump, filters, pipelines, drains, and outlets.
25. Use according to any of the preceding use paragraphs, wherein the Aspergillus fumigatus cellobiohydrolase I or homolog thereof of the enzyme preparation is selected from the group consisting of:
   (i) a cellobiohydrolase I comprising or consisting of the mature polypeptide of SEQ ID NO: 2;
   (ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2;
   (iii) a cellobiohydrolase I encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1; and
   (iv) a cellobiohydrolase I encoded by a polynucleotide that hybridizes under at least high stringency conditions, very high stringency conditions, with the mature polypeptide coding sequence of SEQ ID NO: 1 or the full-length complement thereof;
wherein the Aspergillus fumigatus cellobiohydrolase II or homolog thereof is selected from the group consisting of:
   (i) a cellobiohydrolase II comprising or consisting of the mature polypeptide of SEQ ID NO: 4;
   (ii) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4;
   (iii) a cellobiohydrolase II encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 3; and
   (iv) a cellobiohydrolase II encoded by a polynucleotide that hybridizes under at least high stringency conditions, very high stringency conditions, with the mature polypeptide coding sequence of SEQ ID NO: 3 or the full-length complement thereof;
wherein the Aspergillus fumigatus beta-glucosidase or homolog thereof is selected from the group consisting of:
   (i) a beta-glucosidase comprising or consisting of the mature polypeptide of SEQ ID NO: 6;
   (ii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6;
   (iii) a beta-glucosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5;
   (iv) a beta-glucosidase encoded by a polynucleotide that hybridizes under at least high stringency conditions, very high stringency conditions, with the mature polypeptide coding sequence of SEQ ID NO: 5 or the full-length complement thereof; and
   (v) a beta-glucosidase variant comprising a substitution at one or more positions corresponding to positions 100, 283, 456, and 512 of the mature polypeptide of SEQ ID NO: 6, wherein the variant has beta-glucosidase activity; and
wherein the Penicillium sp. GH61 polypeptide having cellulolytic enhancing activity or homolog thereof is selected from the group consisting of:
   (i) a GH61 polypeptide having cellulolytic enhancing activity comprising or consisting of the mature polypeptide of SEQ ID NO: 6;
   (ii) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
   (iii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
   (iv) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.
26. Use according to any of the preceding use paragraphs, wherein the beta-glucosidase variant of the enzyme preparation comprises one or more (several) substitutions selected from the group consisting of G142S, Q183R, H266Q, and D703G.
27. Use according to any of the preceding use paragraphs, wherein the enzyme preparation further comprises one or more enzymes selected from the group consisting of:
   (i) an Aspergillus fumigatus xylanase or homolog thereof,
   (ii) an Aspergillus fumigatus beta-xylosidase or homolog thereof; or
   (iii) a combination of (i) and (ii);
wherein the Aspergillus fumigatus xylanase or homolog thereof is selected from the group consisting of:
   (i) an Aspergillus fumigatus xylanase comprising or consisting of the mature polypeptide of SEQ ID NO: 10, SEQ ID NO: 12, or SEQ ID NO: 14;
   (ii) a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10, SEQ ID NO: 12, or SEQ ID NO: 14;
   (iii) a xylanase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 9, SEQ ID NO: 11, or SEQ ID NO: 13; and
   (iv) a xylanase encoded by a polynucleotide that hybridizes under at least high stringency conditions, very high stringency conditions, with the mature polypeptide coding sequence of SEQ ID NO: 9, SEQ ID NO: 11, or SEQ ID NO: 13; or the full-length complement thereof; and
wherein the Aspergillus fumigatus beta-xylosidase or homolog thereof is selected from the group consisting of:
   (i) beta-xylosidase comprising or consisting of the mature polypeptide of SEQ ID NO: 16;
   (ii) a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;
   (iii) a beta-xylosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 15; and
   (iv) a beta-xylosidase encoded by a polynucleotide that hybridizes under at least high stringency conditions, very high stringency conditions, with the mature polypeptide coding sequence of SEQ ID NO: 15 or the full-length complement thereof.
28. Use according to any of the preceding use paragraphs comprising the use of an acidic material.
29. A cleaning method for cleaning the interior of a washing machine, which method comprises exposing the interior of the washing machine to one or more enzymes capable of degrading cellulosic material.
30. Cleaning method according to paragraph 29, wherein the one or more enzymes capable of degrading cellulosic material is an enzyme preparation comprising:
   (i) an Aspergillus fumigatus cellobiohydrolase I;
   (ii) an Aspergillus fumigatus cellobiohydrolase II;
   (iii) an Aspergillus fumigatus beta-glucosidase or variant thereof; and
   (iv) a Penicillium sp. GH61 polypeptide having cellulolytic enhancing activity; or homologs thereof.
31. Cleaning method according to any of paragraphs 28-29, wherein the Aspergillus fumigatus cellobiohydrolase I or homolog thereof of the enzyme preparation is selected from the group consisting of:
   (i) a cellobiohydrolase I comprising or consisting of the mature polypeptide of SEQ ID NO: 2;
   (ii) a cellobiohydrolase I comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 2;
   (iii) a cellobiohydrolase I encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 1; and
   (iv) a cellobiohydrolase I encoded by a polynucleotide that hybridizes under at least high stringency conditions, very high stringency conditions, with the mature polypeptide coding sequence of SEQ ID NO: 1 or the full-length complement thereof;
wherein the Aspergillus fumigatus cellobiohydrolase II or homolog thereof is selected from the group consisting of:
   (v) a cellobiohydrolase II comprising or consisting of the mature polypeptide of SEQ ID NO: 4;
   (vi) a cellobiohydrolase II comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 4;
   (vii) a cellobiohydrolase II encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 3; and
   (viii) a cellobiohydrolase II encoded by a polynucleotide that hybridizes under at least high stringency conditions, very high stringency conditions, with the mature polypeptide coding sequence of SEQ ID NO: 3 or the full-length complement thereof;
wherein the Aspergillus fumigatus beta-glucosidase or homolog thereof is selected from the group consisting of:
   (vi) a beta-glucosidase comprising or consisting of the mature polypeptide of SEQ ID NO: 6;
   (vii) a beta-glucosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 6;
   (viii) a beta-glucosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 5;
   (ix) a beta-glucosidase encoded by a polynucleotide that hybridizes under at least high stringency conditions, very high stringency conditions, with the mature polypeptide coding sequence of SEQ ID NO: 5 or the full-length complement thereof; and
   (x) a beta-glucosidase variant comprising a substitution at one or more positions corresponding to positions 100, 283, 456, and 512 of the mature polypeptide of SEQ ID NO: 6, wherein the variant has beta-glucosidase activity; and
wherein the Penicillium sp. GH61 polypeptide having cellulolytic enhancing activity or homolog thereof is selected from the group consisting of:
   (v) a GH61 polypeptide having cellulolytic enhancing activity comprising or consisting of the mature polypeptide of SEQ ID NO: 6;
   (vi) a GH61 polypeptide having cellulolytic enhancing activity comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 8;
   (vii) a GH61 polypeptide having cellulolytic enhancing activity encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 7; and
   (viii) a GH61 polypeptide having cellulolytic enhancing activity encoded by a polynucleotide that hybridizes under at least high stringency conditions, very high stringency conditions, with the mature polypeptide coding sequence of SEQ ID NO: 7 or the full-length complement thereof.
32. Cleaning method according to any of paragraphs 29-31, wherein the beta-glucosidase variant of the enzyme preparation comprises one or more (several) substitutions selected from the group consisting of G142S, Q183R, H266Q, and D703G.
33. Cleaning method according to any of paragraphs 29-32, wherein the enzyme preparation further comprises one or more enzymes selected from the group consisting of:
   (i) an Aspergillus fumigatus xylanase or homolog thereof,
   (ii) an Aspergillus fumigatus beta-xylosidase or homolog thereof; or
   (iii) a combination of (i) and (ii);
wherein the Aspergillus fumigatus xylanase or homolog thereof is selected from the group consisting of:
   (v) an Aspergillus fumigatus xylanase comprising or consisting of the mature polypeptide of SEQ ID NO: 10, SEQ ID NO: 12, or SEQ ID NO: 14;
   (vi) a xylanase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 10, SEQ ID NO: 12, or SEQ ID NO: 14;
   (vii) a xylanase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 9, SEQ ID NO: 11, or SEQ ID NO: 13; and
   (viii) a xylanase encoded by a polynucleotide that hybridizes under at least high stringency conditions, very high stringency conditions, with the mature polypeptide coding sequence of SEQ ID NO: 9, SEQ ID NO: 11, or SEQ ID NO: 13; or the full-length complement thereof; and
wherein the Aspergillus fumigatus beta-xylosidase or homolog thereof is selected from the group consisting of:
   (v) beta-xylosidase comprising or consisting of the mature polypeptide of SEQ ID NO: 16;
   (vi) a beta-xylosidase comprising or consisting of an amino acid sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide of SEQ ID NO: 16;
   (vii) a beta-xylosidase encoded by a polynucleotide comprising or consisting of a nucleotide sequence having at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the mature polypeptide coding sequence of SEQ ID NO: 15; and
   (viii) a beta-xylosidase encoded by a polynucleotide that hybridizes under at least high stringency conditions, very high stringency conditions, with the mature polypeptide coding sequence of SEQ ID NO: 15 or the full-length complement thereof.
34. Cleaning method according to any of paragraphs 29-34, wherein the method comprises exposing the interior of the washing machine to an aqueous solution of an acidic material.
35. Cleaning method according to any of paragraphs 29-35, wherein the method is carried out at the same time as washing a textile in the washing machine.
36. Cleaning method according to paragraph 29-36, wherein the enzyme capable of degrading cellulosic material is comprised in the composition of paragraphs 1-17.
37. Use according to any of claims 1-17, wherein the composition is a laundry detergent composition or a fabric softener composition.
38. Use according to any of claims 1-3, 5-17 and 37, wherein the composition comprises
   (i) an Aspergillus fumigatus cellobiohydrolase I comprising or consisting of the mature polypeptide of SEQ ID NO: 2;
   (ii) an Aspergillus fumigatus cellobiohydrolase II comprising or consisting of the mature polypeptide of SEQ ID NO: 4;
   (iii) an Aspergillus fumigatus beta-glucosidase comprising or consisting of the mature polypeptide of SEQ ID NO: 6; or a variant hereof comprising the following substitutions F100D, S283G, N456E and F512Y; and
   (iv) a Penicillium sp. GH61 polypeptide having cellulolytic enhancing activity comprising or consisting of the mature polypeptide of SEQ ID NO: 8.
39. Method according to paragraph 22, wherein the concentration of the enzyme preparation is in the range of 0.05-4 gram/liter, in the range of 0.1-3 gram/liter, in the range of 0.2-2.5 gram/liter, in the range of 0.2-2 gram/liter or in the range of 0.3-1 gram/liter.
40. Method according to any of paragraphs 18-22, wherein the laundry method is carried out by hand or machine.
41. Method according to any of paragraphs 18-22, wherein the method is carried out at least 5 times.
42. Method according to paragraph 41, wherein the method is carried out at least 10 times, at least 15 times or at least 20 times.

### Laundry Detergent Compositions

### Composition of Ariel Sensitive White & Color, Liquid Detergent Composition:

Aqua, Alcohol Ethoxy Sulfate, Alcohol Ethoxylate, Amino Oxide, Citrid Acid, 012-18 topped palm kernel fatty acid, Protease, Glycosidase, Amylase, Ethanol, 1,2 Propanediol, Sodium Formate, Calcium Chloride, Sodium hydroxide, Silicone Emulsion, Trans-sulphated EHDQ (the ingredients are listed in descending order).

### Brilhante HDL (Unilever, Brazil)

Surfactant (LAS) 7%, Builders 5%, Fillers 86%, Polymers 0.5%, Thickner 1% and fragrance 0.5%. (w/w %)

### Composition of Persil Megaperls (Powder)

15-30% of the following: anionic surfactants, oxygen-based bleaching agent and zeolites, less than 5% of the following: non-ionic surfactants, phosphonates, polycarboxylates, soap, Further ingredients: Perfumes, Hexyl cinnamal, Benzyl salicylate, Linalool, optical brighteners, Enzymes and Citronellol.

### Fabric Softener Compositions

The enzyme preparation of the invention can be comprised in the below mentioned fabric softener compositions and can be used for preventing build-up and/or removal of fuzz and pills from a textile and for improving the whiteness of the textile

### Downy® Fabric Softener Dryer Sheets—all Variesties (Procter & Gamble)

Dipalmethyl Hydroxyethylammoinum Methosulfate, Fatty acid, Polyester Substrate, Clay and Fragrance.

### Gain Original Fresh Fabric Softener

Water, Diethyl ester dimethyl ammonium chloride, Perfume, Calcium Chloride, Formic acid, Dimethicone copolymer, Liquitint™ Green, Hydrochloric Acid, Quaternary acrylate polymer, Ethoxylated cocoalkyl bis(2-hydroxyethyl) methyl ammonium chloride, Perfume microcapsules, Methylchoroisothiazolinone/Methylisothiazolinone, Diethylenetriamine pentaacetate (sodium salt).

### Bamseline Creations Jasmin & Blåbær (Unilever, Denmark)

Aqua, Ditallowoylethyl Hydroxyethylmonium Methosulfate, Isopropyl alcohol, Perfume, Limonene, Butylphenyl Methylpropional, Coumarin, Alpha-Isomethyl Ionone, Polyoxymethylene Melamine, Dimethicone, Imidazolidinone, Benzisothiazolinone, Polymeric Pink Colourant, Etidronic Acid, Trimethylsiloxysilicate, Calcium chloride, Hydrogenated Vegetable Glycerides, Glycol Stearate, Cellulose Gum, Xanthan gum, Polymeric Blue Colourant, lodopropynyl Butylcarbamate.
Ype fabric softener
4% cationic surfactant, 0.3% fatty acid, 0.1% formol 37%, 0.6% perfume, 0.3% color protector, 0.7% thickener and 94% water.

### EXAMPLES

### Enzyme Preparation 2 (Enz 2) Capable of Degrading Cellulosic Material Used in Example 1, 2 and 6.

The enzyme preparation capable of degrading cellulosic material comprises a blend of an Aspergillus fumigatus GH10 xylanase and Aspergillus fumigatus beta-xylosidase with a Trichoderma reesei cellulase preparation containing Aspergillus fumigatus cellobiohydrolase I, Aspergillus fumigatus cellobiohydrolase II, Aspergillus fumigatus beta-glucosidase variant, and Penicillium sp. (emersonii) GH61 polypeptide. The enzyme preparation can be produced as described in examples 1-19 of WO 2013/028928, which is hereby incorporated by reference. This enzyme preparation is different that the enzyme preparation used in example 3, 4 and 5.

### Example 1

Cellulose degrading experiments are performed in order to assess the wash performance—degradation—on cellulose based material like cellulose fibres. One application is to clean the filters and pipes in the laundry washing machine. The cellulose fibres used in this evaluation have been collected from two sources, one from the filter from tumble dryers, in this case Miele SOFTTRONIC T8627 WP with the program Automatic+ and where 75% cotton and 25% polyester had been tumble dried and another one from paper tissues; KIMCARE, Medical Wipes, code 3020—Kimberly-Clark professional.

First the cellulose fibres are acid treated with 10 ml solution of citric acid (6 gram/liter) reaching a pH of about pH 2-3 in a beaker for about 10 minutes. After this, the cellulose fibres are placed on a sieve over a beaker for drying overnight at room temperature. The next day the dry cellulose fibres are placed in a beaker with water at 40° C. and the enzyme preparation 2 capable of degrading cellulosic material with the amount 0.5 gram/liter, are added. After 30 minutes the fibers are taken up and dried again on a filter, the sieve over the beaker, overnight at room temperature. This process is repeated 5 times.

| | Fibres from | Fibres from | | |
|---|---|---|---|---|
| Fiber weight | tumble dryer | tumble dryer | Paper tissue | Paper tissue |
| loss, wt % loss | Test 1 | Test 2 | Test 1 | Test 2 |
| Only water | 18 | 22 | 4 | 9 |
| Enzyme | 35 | 26 | 39 | 42 |
| preparation 2 | | | | |
| capable of | | | | |
| degrading | | | | |
| cellulosic | | | | |
| material | | | | |

### Example 2

### Tensile Strength Evaluation

Tensile strength evaluation is done in order to find out if the enzyme preparation 2 material used will degrade the cotton textiles too much so that it will be negative for a consumer using a commercial product containing the enzyme preparation 2 capable of degrading cellulosic. The tensile strength evaluation was performed at wfk Testgewebe GmbH, Germany. The determination of tensile strength is done according to ISO 13934 T01 and DIN 53919 part 2, see description below. Textile used is wfk 11A, 100% woven cotton.

### Determination of Tensile Strength Loss According to DIN EN ISO 13934-1 and DIN 53919 Part 2

DIN 53919 part 1 describes the fabric construction of the wfk 11A and DIN 53919 part 2 describes how to prepare the specimen for each kind of test. DIN EN ISO 13934-1 describes testing conditions for tensile strength testing.

Repeated wash cycles usually reduce the tensile strength of the cotton control cloth due to the mechanical action and chemical damage during the cycles.

Any decrease in breaking strength expressed as a percentage of the initial breaking strength is determined from the variation in breaking strength measured in the direction of the warp of the control cloth before and after laundering.

The strips for testing tensile strength should be 30 cm (length/warp direction)×6 cm (width). Wfk testgewebe GmbH uses 10 strips (30×6 cm) of the prepared control sheets (see page 3). The strips are cut along the outer green threads (see page 3). After cutting the strips, the outer green warp thread and the white warp threads between the outer and the inner green warp thread have to be pulled out on both sides of the strip; also the inner green warp thread should be pulled out. This leaves exactly 132 warp (white) threads remaining in each single test sample. The width of the samples is approx. 5 cm.

Because the width of the material may change (e.g. due to shrinkage) the number of white threads between the inner green warp threads are very important for comparing the washed values with the initial values.

After separating the 10 strips of one control sheet, the average and the standard deviation of tensile strength values of the control sheet are calculated. If strips tear along the clamps the result of that strips are invalid. If this occurs, new strips of the same laundry control sheet should be tested.

Testing conditions at wfk Testgewebe GmbH for testing tensile strength of laundry control sheet:
distance between the clamps: 200 mm
rate of loading: 100 mm/min
specimen length.30 cm
Amount of load applied to the fabric before starting the tear tester: 1N
testing in warp direction
testing wet (the ten strips of one control sheet are treated in a bath of water with surface active agent for wetting the fabric—wetting time 1 hour)
Number of strips per laundry control sheet minimum 10

The detergent used in this evaluation was model detergent B with two different cellulase preparations and without any enzyme preparation 2 for comparison.

Cellulase preparation 1: the enzyme preparation 2 capable of degrading cellulosic material: 1 wt %.
Cellulase preparation 2: a commercial cellulase of SEQ ID NO:17, 0.13 wt %
Model detergent B consisting of:

### 7% LAS

3% AEOS/SLES—alkyl ethoxylates/Sodium lauryl ether sulfate
6.6% Non-ionic surfactant
5.5% soap
Add up 100% with water
Surfactant content: 16.6%

Model detergent B was dosed with 50 g/wash at 15 dH and 30° C. in Miele Softtronic W2245, washing machine with washing time 1 h 26 minutes. 20 wash cycles were run for each of the two cellulase preparations with tumble drying (Miele SOFTTRONIC T8627 WP with the program Automatic+) in between every wash:

The wash performed without a cellulase preparation is used as baseline in terms of tensile strength.

The results shows the tensile strength of the textile washed with the enzyme preparation 2 capable of degrading cellulosic material gives about 16% strength loss after the 20 washes at whereas the commercial cellulase of SEQ ID NO:17 with gives about 22% strength loss after 20 washes.

The conclusion is therefore that the enzyme preparation 2 capable of degrading cellulosic material is less aggressive towards the wfk 11A textile compared to the commercial cellulase of SEQ ID NO: 17.

### Example 3

### SEQ ID NO: 17 vs Enz 1—Full Scale Wash

This is a multicycle test (20 wash cycles) used to compare performance of SEQ ID NO: 1 versus the enzyme preparation of the invention in full scale wash under Latin American conditions (washing in a top loader washing machine). Fabrics and ballast are added to each wash together with laundry detergent composition and enzymes. After wash, fabrics were line dried. Fabrics appearance was evaluated visually by a group panel.

### Equipment Used:

Washing machine: BWL11A Brastemp

### Ballast

The ballast consists of clean white cloth without optical whitener made of 100% cotton. The ballast weight, dryness and item composition must be the same in each wash. Ballast Example: (Standard LA ballast composition, total 1 kg)
15 pieces 45×45 cm ballast 100% cotton

### Wash Conditions

20 cycle test

### Machine: TOP Load BWL11A Brastemp

Washing temperature (° C.): 25
Washing cycle: cycle “dia-a-dia” (normal)

1 h30 min washing cycle, with two rinsing steps.

Detergent concentration (g/L): 1,8 HDL Brilhante (UL)
Water hardness (ppm): 70 (tap water)
Washing volume (L): 35
Rinsing volume (L): 35
Rinsing time: 15 minutes
Ballast: weight fabrics (see table 1) and complete ballast to 1 kg
Enzyme dosage: see table 2, below:

TABLE 1
Fabrics.

| Fabric | Size | Composition |
|---|---|---|
| Purple striped | 10 pieces 15 × 15 cm | 50/50% cotton/polyester |
| polycotton from | | |
| malharia Fremetax | | |
| Rayon elastane | 10 pieces 15 × 15 cm | 96% rayon/4% elastane |
| from malharia | | |
| Fremetax | | |
| Blue stripped | 10 pieces 15 × 15 cm | 50/50% cotton/polyester |
| polycotton from | | |
| Malharia Fremetex | | |
| Empa 252 from | 10 pieces 10 × 10 cm | Standard cotton from CFT |
| CFT | | |
| Cotton socks from | 10 socks | 100% cotton |
| Hannes | | |

TABLE 2
Enzyme dosage for the test.

| | Laundry Detergent composition | Enzyme % | Enzyme |
|---|---|---|---|
| A | HDL Brilhante | 0.3 | SEQ ID NO: 17 |
| B | HDL Brilhante | 0.5 | SEQ ID NO: 17 |
| C | HDL Brilhante | 0.5 | Enz 1 |
| D | HDL Brilhante | 1.0 | Enz 1 |
| E | HDL Brilhante | 0 | 0 |

Enzyme preparation 1 (Enz 1) comprises:
(i) an Aspergillus fumigatus cellobiohydrolase I comprising or consisting of the mature polypeptide of SEQ ID NO: 2;
(ii) an Aspergillus fumigatus cellobiohydrolase II comprising or consisting of the mature polypeptide of SEQ ID NO: 4;
(iii) an Aspergillus fumigatus beta-glucosidase comprising or consisting of the mature polypeptide of SEQ ID NO: 6; or a variant hereof comprising the following substitutions F100D, S283G, N456E and F512Y; and
(iv) a Penicillium sp. GH61 polypeptide having cellulolytic enhancing activity comprising or consisting of the mature polypeptide of SEQ ID NO: 8.
   Detailed steps to carry out full scale wash trial:
   1) Choose normal cycle (dia-a-dia) at machine, low volume (35L),
   2) Start program to fill up the machines with water and adjust the temperature (25 C) manually.
   3) Add detergent and enzymes/enzyme preparation directly to water (table 2)
   4) Add ballast and fabrics—2 pieces in each machine (table 1)
   5) Close the machine lid and start the cycle.
   6) When the cycle ends (after centrifugation), remove the ballast and separate for the next washing cycle.
   7) Restart from step 1
   8) Run 20 times steps #1 to #7.
   9) After the 20^{th} cycle remove the fabrics and line dry in accordance with procedure.

### Drying Procedure

Hang the fabrics in line and dry at room temperature after the 20^{th} cycle. The room has a de-humidifier working for 24 h per day to keep the room dry

### Measurement

20 trained test persons evaluated the fabric by giving a note from 1 to 10 to each coded sample based on better appearance and renewal of the fibers, were 10 is the best one corresponding to appearance of newly produced fabric. Notes presented are the average to give a single rating number.

Five Fabrics were Tested:
Purple striped polycotton from Malharia Fremetex; Rayon elastane from malharia Fremetax; Cotton socks from Hannes; Empa 252 from CFT; Blue stripped polycotton from Malharia Fremetex.

### Washing Experiment

1.8 g/L of HDL Brilhante was dissolved directly into machine with water hardness of 70 ppm (tap water). In formulas with SEQ ID NO: 17 or Enz 1, enzymes were added based on Table 2 directly to the wash liquor, w/w % from detergent. Furthermore, fabrics plus ballast were added in a total of 1 kg. Add fabrics as prepared on table 1 (2 pieces of each fabric in each machine) and start the program as indicated on washing conditions above. After the 20^{th} washing cycle, fabrics were line dried overnight. Measurement was made using group of people as panelists, given notes from 1 to 10 to each fabric (10 is the best, look as new), based on visual appearance compared to a new one.

### Results

Based on sensory panel analysis, average of rates for each fabric and laundry detergent composition are shown on table 3, below:

TABLE 3
Influence of enzymatic treatment when applied to different fabrics after
20 wash cycles.

| Detergent | Fabric | Enzyme | Rate |
|---|---|---|---|
| HDL | Purple striped polycotton from malharia | 0.3% SEQ ID NO: 17 | 7 |
| Brilhante | Fremetax | | |
| HDL | Purple striped polycotton from malharia | 0.5% SEQ ID NO: 17 | 8.75 |
| Brilhante | Fremetax | | |
| HDL | Purple striped polycotton from malharia | 0.5% Enz 1 | 4.375 |
| Brilhante | Fremetax | | |
| HDL | Purple striped polycotton from malharia | 1% Enz 1 | 7.25 |
| Brilhante | Fremetax | | |
| HDL | Purple striped polycotton from malharia | no enzymes | 1.375 |
| Brilhante | Fremetax | | |
| HDL | Rayon elastane from malharia | 0.3% SEQ ID NO: 17 | 5 |
| Brilhante | Fremetax | | |
| HDL | Rayon elastane from malharia | 0.5% SEQ ID NO: 17 | 6.75 |
| Brilhante | Fremetax | | |
| HDL | Rayon elastane from malharia | 0.5% Enz 1 | 6.5 |
| Brilhante | Fremetax | | |
| HDL | Rayon elastane from malharia | 1% Enz 1 | 6.375 |
| Brilhante | Fremetax | | |
| HDL | Rayon elastane from malharia | no enzymes | 1.75 |
| Brilhante | Fremetax | | |
| HDL | Blue stripped polycotton from Malharia | 0.3% SEQ ID NO: 17 | 6.625 |
| Brilhante | Fremetex | | |
| HDL | Blue stripped polycotton from Malharia | 0.5% SEQ ID NO: 17 | 7.5 |
| Brilhante | Fremetex | | |
| HDL | Blue stripped polycotton from Malharia | 0.5% Enz 1 | 5.5 |
| Brilhante | Fremetex | | |
| HDL | Blue stripped polycotton from Malharia | 1% Enz 1 | 7.875 |
| Brilhante | Fremetex | | |
| HDL | Blue stripped polycotton from Malharia | no enzymes | 1.375 |
| Brilhante | Fremetex | | |
| HDL | Empa 252 from CFT | 0.3% SEQ ID NO: 17 | 6.875 |
| Brilhante | | | |
| HDL | Empa 252 from CFT | 0.5% SEQ ID NO: 17 | 6.25 |
| Brilhante | | | |
| HDL | Empa 252 from CFT | 0.5% Enz 1 | 5.75 |
| Brilhante | | | |
| HDL | Empa 252 from CFT | 1% Enz 1 | 7.125 |
| Brilhante | | | |
| HDL | Empa 252 from CFT | no enzymes | 1.625 |
| Brilhante | | | |
| HDL | Cotton socks from Hannes | 0.3% SEQ ID NO: 17 | 7.87 |
| Brilhante | | | |
| HDL | Cotton socks from Hannes | 0.5% SEQ ID NO: 17 | 7.75 |
| Brilhante | | | |
| HDL | Cotton socks from Hannes | 0.5% Enz 1 | 6.625 |
| Brilhante | | | |
| HDL | Cotton socks from Hannes | 1% Enz 1 | 6.375 |
| Brilhante | | | |
| HDL | Cotton socks from Hannes | no enzymes | 1.875 |
| Brilhante | | | |

### Example 4

### Enzyme Preparation 1—Full Scale Wash

This is a multicycle test (20 wash cycles) used to prove performance (softness and fiber care) of Enzyme preparation 1 (Enz 1) in fabric softeners on full scale wash under latin american conditions (washing in a top loader washing machine). Fabrics and ballast are added to each wash together with laundry detergent composition, fabric softeners and enzymes. After wash, fabrics were line dried. Fabrics appearance was evaluated visually by a group panel.

The equipment used, the ballast and the wash conditions are the same as in example 3.

TABLE 4
Fabrics.

| Fabric | Size | Composition |
|---|---|---|
| Blue stripped | 10 pieces 15 × 15 cm | 50/50% cotton/polyester |
| polycotton | | |
| from Malharia | | |
| Fremetex | | |
| Rayon elastane | 10 pieces 15 × 15 cm | 96% rayon/4% elastane |
| from malharia | | |
| Fremetax | | |
| Cotton towel | 10 pieces 20 × 20 cm | 200 g/cm2 cotton towel |
| from Budmeyer | | |
| Empa 252 from | 10 pieces 10 × 10 cm | Standard cotton from CFT |
| CFT | | |

TABLE 5
Enzyme dosage for the test.

| | | Fabric | | |
|---|---|---|---|---|
| | Laundry Detergent Composition | Softener | Enzyme % | Enzyme |
| A | HDL Brilhante | Ype | 0.5 | Enz 1 |
| B | HDL Brilhante | Ype | 2.5 | Enz 1 |
| C | HDL Brilhante | Ype | 5.0 | Enz 1 |
| D | HDL Brilhante | Ype | 0 | — |

### Enzyme Preparation 1 (Enz 1): See Details of Composition in Example 3.

### Detailed Steps to Carry Out Full Scale Wash Trial:

1) Choose normal cycle (dia-a-dia) at machine, low volume (35L), and add an extra rinsing step for softeners.
2) Start program to fill up the machines with water and adjust the temperature (25 C) manually.
3) Add detergent directly to water.
4) Weigh Softeners and Enzyme preparation (table 5) and mix them together on softener special compartment of machine (the softener will be delivered only during the second rinsing step)
5) Add ballast and fabrics—2 pieces in each machine (table 4)
6) Close the machine lid and start the cycle.
7) When the cycle ends (after centrifugation), remove the ballast and separate for the next washing cycle.
8) Restart from step 1
9) Run 20 times steps #1 to #7.
10) After the 20^{th} cycle remove the fabrics and line dry in accordance with procedure.

### Drying Procedure

Hang the fabrics in line and dry at room temperature after the 20^{th} cycle. The room has a de-humidifier working for 24 h per day to keep the room dry

### Measurement

20 trained test persons evaluated the fabric by giving a note from 1 to 10 to each coded sample based on better appearance (renewal of the fibers) and softness for the towels, were 10 is the best one corresponding to appearance and softness of newly produced fabric. Notes presented are the average to give a single rating number.

Four Fabrics were Tested:
Blue stripped polycotton from Malharia Fremetex.; Rayon elastane from malharia Fremetax; Cotton towel from Budmeyer; Empa 252 from CFT;

### Washing Experiment

1.8 g/L of HDL Brilhante was dissolved directly into machine with water hardness of 70 ppm (tap water). At formulas with Enz 1, enzymes were added together with softener into softener special compartment on the machine, based on Table 5, w/w % from softener. Furthermore, fabrics plus ballast were added in a total of 1 kg, as prepared on table 4 (2 pieces of each fabric in each machine) and start the “dia a dia” program, following the steps described on washing conditions. After the 20^{th} washing cycle, fabrics were line dried overnight. Measurement was made using group of people as panelists, given notes from 1 to 10 to each fabric (10 is the best, look as new), based on visual appearance and softness compared to a new one.

### Results

Based on sensory panel analysis, average of rates for each fabric and formula are shown on table 6 (for appearance) and table 7 (for softness), below:

TABLE 6
rates of appearance when enzymes are applied.

| Laundry | | | | |
|---|---|---|---|---|
| Detergent | Fabric | | | |
| composition | Softener | Fabric | Enzyme | Rate |
| HDL Brilhante | Ype | All fabrics | 0.5% Enz 1 | 3.5 |
| HDL Brilhante | Ype | All fabrics | 2.5% Enz 1 | 5.79 |
| HDL Brilhante | Ype | All fabrics | 5% Enz 1 | 9.3 |
| HDL Brilhante | Ype | All fabrics | 0 | 2.13 |

TABLE 7
rates of softness when enzymes are applied.

| Laundry | | | | |
|---|---|---|---|---|
| Detergent | Fabric | | | |
| composition | Softener | Fabric | Enzyme | Rate |
| HDL Brilhante | Ype | All fabrics | 0.5% Enz 1 | 4.22 |
| HDL Brilhante | Ype | All fabrics | 2.5% Enz 1 | 6.41 |
| HDL Brilhante | Ype | All fabrics | 5% Enz 1 | 9.8 |
| HDL Brilhante | Ype | All fabrics | 0 | 4.16 |

### Example 5

Exposure Test with Enz 1—Full Scale Wash

This is a shaking test used to prove safety for end user of Enz 1 applied to a fabric softener formulation, on full scale wash under LA conditions (washing in a top loader washing machine). T-shirts were added to wash together with detergent, softeners and enzymes. After wash, t-shirts were line dried.

### Equipment Used:

Washing machine: BWL11A Brastemp

### Wash Conditions

1 cycle wash

### Machine: TOP Load BWL11A Brastemp

Washing temperature (° C.): 25
Washing cycle: cycle “dia-a-dia” (normal) 1 h30 min washing cycle, with two rinsing steps. Softeners+enzymes added on second rinsing step.
Detergent concentration (g/L): 1,8 HDL Brilhante (UL)
Water hardness (ppm): 70 (tap water)
Washing volume (L): 75 (high)
Rinsing volume (L): 75 (high)
Rinsing time: 15 minutes
Agitation speed (rpm): 100 rpm
Ballast: 2 kg of tshirts—table 8
Enzyme Dosages: table 9
Measurement: ELISA assay

### Softener: 50 g

TABLE 8
Fabrics.

| Fabric | # | Composition |
|---|---|---|
| Hering super cotton t-shirt | 30 t-shirts | 100% cotton |

TABLE 9
Enzyme dosage for the test.

| | Detergent | Softener | Enzyme % | Enzyme Name |
|---|---|---|---|---|
| A | HDL Brilhante | Ype | 0.5 | Enz 1 |
| B | HDL Brilhante | Ype | 2.5 | Enz 1 |
| C | HDL Brilhante | Ype | 5.0 | Enz 1 |

### Enzyme Preparation 1 (Enz 1): See Details of Composition in Example 3.

### Detailed Steps to Carry Out Full Scale Wash Trial:

1) Choose normal cycle (dia-a-dia) at machine, low volume (35L), and add an extra rinsing step for softeners.
2) Start program to fill up the machines with water and adjust the temperature (25 C) manually.
3) Add detergent directly to water.
4) Weigh Softeners and Enzymes (table 9) and mix them together on softener special compartment of machine (the softener will be delivered only during the second rinsing step)
5) Add t-shirts—10 pieces in each machine (table 8)
6) Close the machine lid and start the cycle.
7) When the cycle ends (after centrifugation), remove the t-shirts and line dry in accordance with procedure.

### Drying Procedure

Hang the t-shirts in line and dry at room temperature after the 20^{th} cycle. The room has a de-humidifier working for 24 h per day to keep the room dry

### Washing Experiment

1.8 g/L of HDL Brilhante was dissolved directly into machine with water hardness of 70 ppm (tap water). Enzymes were added together with softener into softener special compartment on the machine, based on Table 9, w/w % from softener. Furthermore, t-shirts were added in a total of 2 kg, as prepared on table 8 (10 pieces of t-shirts in each machine) and start the “dia a dia” program, following the steps described on washing conditions. After the washing cycle, t-shirts were line dried overnight.

### Example 6

### Full Scale Wash

This is the test method used to test the wash performance of enzyme preparations in full scale wash under EU conditions (washing in a front loader washing machine). For wash cycles with SEQ ID NO: 17 tea towel with swatches of EMPA 252 and pre-aged EMPA 252 (washed 6 hours) in addition to laundry control sheet WFK 11A, WFK 80A were added to each wash cycle with liquid model detergent, enzymes and soil. After each wash cycle tea towels with swatches of EMPA 252 were tumble dried between every wash cycle dried overnight on filter paper and remission was measured at 460 nm as described above. The L values of the black color of EMPA 252. For wash cycles with SEQ ID NO: 18 swatches (6/wash cycle) of CFT (CN-11, CN-42, CT-01, PCN-01, PN-01), EMPA 221, WFK (12AW, 20A, 30A, 80A) and knitted cotton t-shirt (2/wash cycle) were added. Soil donor (WFK greying swatch I, ‘wfk sock’, wfk Testgewebe GmbH) was added to each wash. Swatches were not tumble dried between every wash cycle.

### Equipment Used:

Washing machine: Miele Softtronic W2445
Water meters and automatically data collection system
(L value is a color eye measurement without UV)

### Equipment Used for Pre-Aging:

Wascator FOM 71 CLS (supplied by SDL Atlas) program 150, 6 Hour, 40° C., 21 L water, 15 dH

### For the Preparation and Adjustment of Water Hardness the Following Ingredients are Needed:

Calcium chloride (CaCl₂.2H₂O)
Magnesium chloride (MgCL₂.6H₂O)
Sodium Hydrogen Carbonate (NaHCO₃)

### Laundry Detergent Composition

| Liquid Model detergent | wt % |
|---|---|
| LAS | 7.2 |
| AEOS(SLES) | 4.2 |
| Soap-cocoa | 2.75 |
| Soap-soya | 2.75 |
| AEO biosoft N25-7 | 6.6 |
| NAOH | 1.2 |
| Ethanol | 3 |
| MPG | 6 |
| glycerol | 2 |
| Tea | 3 |
| Sodium formiate | 1 |
| sodium citrate | 2 |
| DMPA | 0.2 |
| PCA | 0.2 |
| ion exchanged water | 55.08 |

### Ballast

The ballast consists of clean white cloth without optical whitener made of cotton, polyester or cotton/polyester. The composition of the ballast is a mix of different items at a cotton/polyester ratio of 65/35 based on weight. The ballast weight, dryness and item composition must be the same in each wash cycle.

After each wash cycle the ballast is inactivated in an industrial washer at 85° C./15 min or in a 95° C. wash (EU machine) without detergent

Ballast Example: (Standard EU ballast composition, total 3 kg)
2 T-shirts (100% cotton)
5 shirts, short sleeves (55% cotton 45% polyester)
1 pillow cases (35% cotton, 65% polyester), 110×75 cm
1 small bed sheets, size 100×75 cm (100% cotton)
Socks (80% cotton 20% polyester) as balance

### Wash Conditions

Temperature: 30° C.
Washing programme: Normal cotton wash without pre-wash: “Cottons”.
Water level 13-14L with “water plus”
Water hardness: Standard EU conditions: 15° dH, Ca2+:Mg2+:HCO3=4:1:7.5
Various cellulose dosages (SEQ ID NO: 17 0.13 wt % (=0,731 mg enzyme protein (EP)/wash cycle) and 0.26 wt % (=1.46 mg EP/wash), Enzyme preparation 2 1 wt % (=60 mg EP/wash cycle)
Liquid model detergent dosage 50 g/wash cycle

### Detailed Steps to Carry Out Full Scale Wash Trial

1. Select wash program as in study plan.
2. The detergent and cellulase are placed in the wash drum in a “washing ball” (both liquid and powder detergents). Place it at the bottom.
3. Place the tea towels with swatches and and ballast in the wash drum.
4. Start digital water meter
5. Start the washer by pressing the knob START
6. After wash, take out teatowels with swatches and ballast, dry items.
Repeat the above procedure up to 20 times.

TABLE 10
Whiteness effect is determined by measuring remission on WFK 11A,
WFK 80A at 460 nm as described above. Remission after 3 and 5 wash
cycles of SUM of 10 white fabrics (WFK 11A, WFK 80A). The higher
remission value, the whiter the fabric.
Conclusion: Some whiteness effect with 1% enzyme preparation 2 but not
as good as SEQ ID NO: 18.

| SUM of all (10 white swatches/T-shirt) | 460 nm | 460 nm |
|---|---|---|
| Laundry detergent composition and enzyme | 3 wash | 5 wash |
| | cycles | cycles |
| Liquid model detergent | 786 | 766 |
| Liquid model detergent + 0.13% SEQ ID NO: 18 | 830 | 819 |
| Liquid model detergent + 0.26% SEQ ID NO: 18 | 836 | 820 |
| Liquid model detergent + 1.00% Enzyme preparation 2 | 813 | 799 |
| Liquid model detergent + 1.00% Enzyme preparation | 820 | 806 |
| 2 + 0.13% SEQ ID NO: 18 | | |

TABLE 11
Prevention of pilling. The lower value, the better anti-pilling effect.

| | | L-value | 5 | 10 | 15 | 20 |
|---|---|---|---|---|---|---|
| | | Before | wash | wash | wash | wash |
| condition | EMPA 252 - not pre-aged | wash | cycles | cycles | cycles | cycles |
| 1 | Liquid model detergent | 20.3 | 26.0 | 30.2 | 31.3 | 32.2 |
| 2 | Liquid model detergent + | 20.3 | 26.4 | 29.0 | 28.8 | 28.2 |
| | 0.13% SEQ ID NO: 17 | | | | | |
| 3 | Liquid model detergent + | 20.3 | 25.7 | 28.1 | 27.1 | 26.7 |
| | 0.26% SEQ ID NO: 17 | | | | | |
| 7 | Liquid model detergent + | 20.3 | 26.0 | 28.6 | 30.6 | 30.3 |
| | 1.00% Enzyme preparation 2 | | | | | |

TABLE 12
removal of fuzz and pills at aged textile (Rejuvenation) (pre-aged EMPA 252).

| | | L-value | 5 | 10 | 15 | 20 |
|---|---|---|---|---|---|---|
| | | Before | wash | wash | wash | wash |
| condition | Pre-aged EMPA 252 | wash | cycles | cycles | cycles | cycles |
| 1 | Liquid model detergent | 32.31 | 32.57 | 33.60 | 33.64 | 33.61 |
| 2 | Liquid model detergent + | 32.31 | 32.12 | 30.61 | 29.06 | 27.92 |
| | 0.13% SEQ ID NO: 17 | | | | | |
| 3 | Liquid model detergent + | 32.31 | 31.11 | 28.49 | 27.07 | 26.39 |
| | 0.26% SEQ ID NO: 17 | | | | | |
| 7 | Liquid model detergent + | 32.31 | 32.77 | 31.96 | 30.66 | 30.07 |
| | 1.00% Enzyme preparation 2 | | | | | |

### Wash Performance ΔL, Δa and Δb

Wash performance can be expressed as Lab color vector (ΔL). After washing and rinsing the swatches were spread out flat and allowed to air dry at room temperature overnight. All washes are evaluated the day after the wash. To evaluate the specific nature of the cleaning, the CIE L*, a* and b* values were also recorded by the Macbeth Color Eye 7000 reflectance spectrophotometer during the measurement. The Lab color measurements taken using the Color Eye 7000 are calculated from the CIE (Commission internationale de l'eclairage) XYZ color space co-ordinates. Lab is the abbreviation used to describe the CIE 1976 L*, a*, b* color space, where L is lightness and a, and b are color dimensions.

ΔL denotes the change in L* when taken the measurements from swatches washed with the enzyme of the invention and subtract with the measurements from swatches washed without enzyme for each stain.
Δa denotes the change in a* when taken the measurements from swatches washed with the enzyme of the invention and subtract with the measurements from swatches washed without enzyme for each stain.
Δb denotes the change in b* when taken the measurements from swatches washed with the enzyme of the invention and subtract with the measurements from swatches washed without enzyme for each stain.
Reference is made to János Schanda (2007). Colorimetry. Wiley-Interscience. p. 61. ISBN 978-0-470-04904-4.

1-6. (canceled)

7. A method for laundering a textile, comprising the steps of:
a) contacting the textile with a wash liquor comprising a laundry detergent composition;
b) completing at least one wash cycle;
c) contacting the textile with water comprising a fabric softener composition; and
d) completing at least one rinse cycle;
wherein the laundry detergent composition or the fabric softener composition comprise an enzyme preparation comprising one or more enzymes capable of degrading cellulosic material and wherein the one or more enzymes capable of degrading cellulosic material comprises:
i. an Aspergillus fumigatus cellobiohydrolase I;
ii. an Aspergillus fumigatus cellobiohydrolase II;
iii. an Aspergillus fumigatus beta-glucosidase or variant thereof; and
iv. a Penicillium sp. GH61 polypeptide having cellulolytic enhancing activity; or homologs thereof; and
wherein the method prevents build-up or removes fuzz and pills from the textile and improves the whiteness of the textile.

8. The method according to claim 7, wherein the laundry detergent composition or the fabric softener composition comprises:
(iv) an Aspergillus fumigatus cellobiohydrolase I comprising the mature polypeptide of SEQ ID NO: 2;
(v) an Aspergillus fumigatus cellobiohydrolase II comprising the mature polypeptide of SEQ ID NO: 4;
(vi) an Aspergillus fumigatus beta-glucosidase comprising the mature polypeptide of SEQ ID NO: 6; or a variant hereof comprising the following substitutions F100D, S283G, N456E, F512Y, and
(vii) a Penicillium sp. GH61 polypeptide having cellulolytic enhancing activity comprising the mature polypeptide of SEQ ID NO: 8.

9. The method according to claim 7, wherein the method is carried out by hand or machine.

10. The method according to claim 7, wherein the method is carried out at least 5 times.

11. The method according to claim 10, wherein the method is carried out at least 10 times.

12. The method according to claim 7, wherein a concentration of the enzyme preparation is in the range of 0.03 to 5 gram/liter.

13. The method according to claim 12, wherein the concentration of the enzyme preparation is in the range of 0.05-4 gram/liter.

14. A cleaning method for cleaning an interior of a washing machine, which method comprises exposing the interior of the washing machine to one or more enzymes capable of degrading cellulosic material, wherein the one or more enzymes capable of degrading cellulosic material is an enzyme preparation comprising:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or
homologs thereof.

15. The method of claim 7, wherein the Aspergillus fumigatus cellobiohydrolase I comprises an amino acid sequence having at least 70% sequence identity to the mature polypeptide of SEQ ID NO: 2.

16. The method of claim 7, wherein the Aspergillus fumigatus cellobiohydrolase II comprises an amino acid sequence having at least 70% identity to the mature polypeptide of SEQ ID NO: 4.

17. The method of claim 7, wherein the Aspergillus fumigatus beta-glucosidase is selected from the group consisting of:
(i) an amino acid sequence having at least 70% sequence identity to the mature polypeptide of SEQ ID NO: 6; and
(ii) a variant comprising a substitution at one or more positions corresponding to positions 100, 283, 456, and 512 of the mature polypeptide of SEQ ID NO: 6, wherein the variant has beta-glucosidase activity.

18. The method of claim 7, wherein the Penicillium sp. GH61 polypeptide or homolog comprises an amino acid sequence having at least 70% sequence identity to the mature polypeptide of SEQ ID NO: 8.

19. The method of claim 7, wherein the enzyme preparation further comprises enzymes selected from the group consisting of:
(i) an Aspergillus fumigatus xylanase having an amino acid sequence at least 70% identical to the mature polypeptide of SEQ ID NO: 10, SEQ ID NO: 12, or SEQ ID NO: 14;
(ii) an Aspergillus fumigatus beta-xylosidase having an amino acid sequence at least 70% identical to the mature polypeptide of SEQ ID NO: 16; and
(iii) a combination of (i) and (ii).

20. The method of claim 7, wherein the laundry detergent composition or the fabric softener composition comprises at least one additional enzyme selected from the group consisting of hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, ß-glucanases, arabinosidases, hyaluronidase, chondroitinase, laccase, DNases, chlorophyllases, amylases, perhydrolases, peroxidases, and xanthanase.
